(19) United States
(12) Patent Application Publication
(10) Pub. No.: US 2022/0331223 A1
(43) Pub. Date: Oct. 20, 2022

(54) HAIR TREATMENT COMPOSITIONS AND METHODS OF USE

(71) Applicant: VIRTUE LABS, LLC, Winston Salem, NC (US)

(72) Inventors: Erin FALCO, Winston Salem, NC (US); William JACOBSEN, Clemmons, NC (US); Marc REINER, New York, NY (US); Debby VELLOZZI, Stamford, CT (US); Emily Anne SHARPE, Winston Salem, NC (US)

(21) Appl. No.: 17/720,507

(22) Filed: Apr. 14, 2022

Related U.S. Application Data
(60) Provisional application No. 63/174,875, filed on Apr. 14, 2021.

(51) Int. Cl. A61K 8/65; A61K 8/64; A61K 8/9789; A61K 8/67; A61K 8/9794; A61K 8/9717; A61K 8/73; A61Q 7/00; A61Q 19/00

(52) U.S. Cl. CPC A61K 8/65; A61K 8/64; A61K 8/9789; A61K 8/673; A61K 8/9794; A61K 8/9717; A61K 8/735; A61K 8/675; A61Q 7/00; A61Q 19/00

(57) ABSTRACT

Hair treatment compositions are provided that include alpha keratin and gamma keratin proteins derived from human hair, at least one biomimetic signal peptide, and red clover extract. Methods of treatment are also provided.

### CROSS-REFERENCE TO RELATED APPLICATIONS

The present application claims priority to U.S. Ser. No. 63/174,875 filed Apr. 14, 2021, the contents of which is incorporated herein in its entirety.

### BACKGROUND

The hair growth cycle is made up of three distinct phases (anagen, catagen and telogen). The anagen phase is the growth phase; catagen is the regression/follicle contraction phase; and telogen is the resting/fall out phase. Hair loss may occur when the normal balance between growth phase hair and rest phase is changed and a telogen phase predominates. Disproportional hair loss may lead to the decrease of the total number of the strands of hair. Hair loss may have no specific cause (e.g., syphilitic alopecia). In other cases, hair loss may be caused by physiological stress, surgical trauma, high fever, hemorrhage, changes in thyroidal hormones, reduction of the levels of estrogen after gestation, metabolic or hormonal disturbances, nutritional factors, and medications (e.g., chemotherapy, anti-thyroidal agents, hormones, anticonvulsants, anticoagulants, beta-blockers, inhibitors of angiotensin and lithium conversing enzyme).

Together with the elimination of the cause which triggered the hair loss, treatments of hair loss include stimulation of the follicular metabolism in the form of replacement therapy. The appropriate operation of the cell metabolism increases the resistance of the hair to new external issues and it stimulates the healthy hair growth. Such current treatments often require the use of harsh active pharmaceutical ingredients such as corticosteriods or immune suppressants.

### SUMMARY

A hair treatment composition is provided. The hair treatment composition includes a keratin protein composition including a blend of alpha keratin and gamma keratin. The hair treatment may further include at least one peptide. The hair treatment may further include a clover extract such as red clover extract. According to one embodiment, the hair treatment composition includes a complex that includes the keratin protein composition, at least one peptide, and at least one clover extract. According to one embodiment, the hair treatment composition includes a complex that consists of the keratin protein composition, at least one peptide, and at least one clover extract.

The hair treatment compositions provided herein may be formulated as a shampoo, conditioner, spray, foam, scalp care composition, aqueous solution, powder, lotion, hydrogel, oil, emulsion, paste, polish or cream. The hair treatment compositions as provided herein are particularly suited for the uses and methods of treatment described herein including, but not limited to, supporting or promoting an increased percentage of anagen hair, increasing or otherwise promoting hair density, providing a noticeable improvement of new hair such as by increasing or promoting the percentage of anagen hair, decreasing thinning hair, reducing hair shedding, nourishing the scalp, increasing scalp pH, maintaining scalp microbiome diversity, and improving scalp skin flora ecology.

### BRIEF DESCRIPTION OF THE DRAWINGS

FIG. 1A illustrates the baseline anagen hair count (hair count was 138) of an exemplary test subject at initial assessment (T0-D0).

FIG. 1B illustrates the anagen hair count (hair count was 216; increase of 78 over baseline) of the same test subject at 120 days (T120).

FIG. 1C illustrates the anagen hair count (hair count was 248; increase of 110 over baseline) of the same test subject at 180 days.

FIG. 2A provides whole head photographic evidence of hair loss of an exemplary test subject at initial assessment (T0-D0).

FIG. 2B provides whole head photographic evidence supporting improved hair growth and density of the same exemplary test subject in FIG. 2A at 120 days (T120).

FIG. 2C provides whole head photographic evidence supporting improved hair growth and improved hair density of the same exemplary test subject in FIG. 2A at 180 days (T180).

FIG. 3A provides whole head photographic evidence of hair loss of an exemplary test subject at initial assessment (T0-D0).

FIG. 3B provides whole head photographic evidence supporting further improved hair growth and improved hair density of the same exemplary test subject in FIG. 3A at 180 days (T180).

### DETAILED DESCRIPTION

One or more aspects and embodiments may be incorporated in a different embodiment although not specifically described. That is, all aspects and embodiments can be combined in any way or combination. When referring to the compounds disclosed herein, the following terms have the following meanings unless indicated otherwise. The following definitions are meant to clarify, but not limit, the terms defined. If a particular term used herein is not specifically defined, such term should not be considered indefinite. Rather, terms are used within their accepted meanings.

The term “keratin protein source” includes proteinaceous sources of keratin proteins from human hair. The human hair can be end-cut, as one would typically find in a barber shop or salon.

The term “keratin protein(s)” as used herein collectively refers to hair protein sources, including, but not limited to, naturally occurring keratin, reduced keratin, oxidized keratin, S-sulfonated keratin, or a combination thereof. This term also refers to the extracted keratin derivatives that are produced by oxidative and/or reductive treatment of keratin, including but not limited to alpha keratin and gamma keratin. Soluble keratins can be extracted from human hair fibers by oxidation or reduction using methods known in the art.

The term “alpha keratin” as used herein refers to a type of keratin protein. Alpha keratin includes the keratin protein that is a large, spring-shaped protein. Alpha keratin as provided herein nourishes or otherwise treats and improves the health of the scalp microbiome, repairs damage along the hair fiber, and reinforces hair strength. The alpha keratin may vary in molecular weight but typically falls in a range of from about 60 Da to about 6.2 MDa. The alpha keratin may be present as a whole, human keratin protein similar or substantially identical in protein conformation to native alpha keratin protein found in hair, skin and nails.

The term “gamma keratin” as used herein refers to a type of keratin protein that includes small, globular proteins that treat hair to provide elasticity and flexibility to hair. The gamma keratins provided herein also aid in the treatment and prevention of hair breakage (i.e., hair breakage reduction) and, when combined with alpha keratin, aids in the treatment and prevention of hair loss (e.g. hair fall-out). The gamma protein may maintain a protein conformation that is similar or identical to the native gamma keratin protein found in hair, skin and nails.

The term “molecular weight” as used herein refers to peak average molecular weight (Mp) which may be determined by methods known in the art such as through high performance liquid chromatography.

The terms “hair” and “mammalian hair” refer to hair on any part of the body of a mammal, and can include but is not limited to facial, cranial, or body hair and can further include hair on the scalp, head, neck, beard, moustache, eyebrows and sideburns.

The term “effective amount,” as used herein, means an amount of a hair treatment composition sufficient to attain the intended result.

The terms “hair thinning” and “hair loss” as used herein may be used interchangeably and encompass any absence or diminishing of hair on the skin of a mammal.

The term “alopecia” refers to and encompasses the various types of alopecia conditions including syphilitic alopecia, alopecia areata, alopecia totalis, alopecia universalis, diffuse alopecia areata, telogen effluvium, cicatricial alopecia, traumatic alopecia, androgenetic alopecia and ophiasis alopecia.

The term “anagen hair” refers to hair in the anagen growing phase which is the initial phase of hair growth from when hair is pushed out of hair follicles until the hair is cut or falls out (i.e., end of life cycle).

The term “telogen hair” refers to hair in the third phase of the hair growth cycle where hair ceases to grow and rests until falling out.

### Hair Treatment Composition

Hair treatment compositions are provided. The hair treatment compositions as provided herein are particularly suited for the uses and methods of treatment described herein including, but not limited to, supporting or promoting hair growth, increasing or otherwise promoting hair density, providing a noticeable growth of new hair, decreasing thinning hair, and reducing hair shedding.

According to one embodiment, the hair treatment composition includes a keratin protein composition as provided herein. According to one embodiment, the keratin protein composition includes both alpha keratin protein and gamma keratin protein. According to one embodiment, the keratin protein composition consists essentially of alpha keratin protein and gamma keratin protein. According to one embodiment, the keratin protein composition consists of alpha keratin protein and gamma keratin protein. According to one embodiment, the keratin protein composition is substantially viscous at both ambient temperature and at about 3° C.

According to one embodiment, the hair treatment compositions provided herein include about 10% w/w or less of keratin protein composition based on the total weight percent of the hair treatment composition. According to one embodiment, the hair treatment compositions provided herein include about 9% w/w or less of keratin protein composition. According to one embodiment, the hair treatment compositions provided herein include about 8% w/w or less of keratin protein composition. According to one embodiment, the hair treatment compositions provided herein include about 7% w/w or less of keratin protein composition. According to one embodiment, the hair treatment compositions provided herein include about 6% w/w or less of keratin protein composition. According to one embodiment, the hair treatment compositions provided herein include about 5% w/w or less of keratin protein composition. According to one embodiment, the hair treatment compositions provided herein include about 4% w/w or less of keratin protein composition. According to one embodiment, the hair treatment compositions provided herein include about 3% w/w or less of keratin protein composition. According to one embodiment, the hair treatment compositions provided herein include about 2% w/w or less of keratin protein composition. According to one embodiment, the hair treatment compositions provided herein include about 1% w/w or less of keratin protein composition. According to one embodiment, the hair treatment compositions provided herein include about 0.1% w/w or less of keratin protein composition. According to one embodiment, the hair treatment compositions provided herein include about 0.1% w/w to about 10% w/w of keratin protein composition. According to one embodiment, the hair treatment compositions provided herein include about 1% w/w to about 7% w/w of keratin protein composition.

According to one embodiment, the keratin protein composition includes alpha keratin in an amount of at least about 1%, 5%, 10%, 15%, 20%, 25%, 30%, 35%, 40%, 45%, 50%, 55%, 60%, 65%, 70%, 75%, 80%, 85%, 90%, 91%, 92%, 93%, 94%, 95%, 96%, 97%, 98%, or 99% by weight (w/w %) based on the total keratin in the keratin protein composition. According to one embodiment, the keratin protein composition includes gamma keratin in an amount of at least about 1%, 2%, 3%, 4%, 5%, 6%, 7%, 8%, 9%, 10%, 15%, 20%, 25%, 30%, 35%, 40%, 45%, 50%, 55%, 60%, 65%, 70%, 75%, 80%, 85%, 90%, 95% or 99% by weight (w/w %) based on the total keratin in the keratin protein composition.

According to one embodiment, the keratin protein composition includes alpha keratin in an amount of at least about 90% w/w to about 99% w/w and gamma keratin in an amount of at least about 1% w/w to about 10% w/w based on the keratin in the keratin protein composition. According to one embodiment, the keratin protein composition includes alpha keratin in an amount of at least about 92% w/w to about 98% w/w and gamma keratin in an amount of at least about 2% w/w to about 8% w/w based on the keratin in the keratin protein composition. According to one embodiment, the keratin protein composition includes alpha keratin in an amount of at least about 93% w/w to about 97% w/w and gamma keratin in an amount of at least about 3% w/w to about 7% w/w based on the keratin in the keratin protein composition. According to one embodiment, the keratin protein composition includes alpha keratin in an amount of at least about 94% w/w to about 96% w/w and gamma keratin in an amount of at least about 4% w/w to about 6% w/w based on the keratin in the keratin protein composition. According to one embodiment, the keratin protein composition includes alpha keratin in an amount of at least about 95% w/w and gamma keratin in an amount of at least about 5% w/w based on the keratin in the keratin protein composition.

According to one embodiment, the keratin protein composition exhibits a peak average molecular weight (Mp) of at least about 443 kDa. According to one embodiment, the alpha keratin of the keratin protein composition exhibits a peak average molecular weight (Mp) of at least about 500 kDa. According to one embodiment, the alpha keratin of the keratin protein composition exhibits a peak average molecular weight (Mp) of at least about 600 kDa. According to one embodiment, the alpha keratin of the keratin protein composition exhibits a peak average molecular weight (Mp) of at least about 700 kDa. According to one embodiment, the alpha keratin of the keratin protein composition exhibits a peak average molecular weight (Mp) of at least about 800 kDa. According to one embodiment, the alpha keratin of the keratin protein composition exhibits a peak average molecular weight (Mp) of at least about 850 kDa. According to one embodiment, the alpha keratin of the keratin protein composition exhibits a peak average molecular weight (Mp) of at least about 900 kDa. According to one embodiment, the alpha keratin of the keratin protein composition exhibits a peak average molecular weight (Mp) of at least about 1.0 MDa. According to one embodiment, the alpha keratin of the keratin protein composition exhibits a peak average molecular weight (Mp) of at least about 1.1 MDa. According to one embodiment, the alpha keratin of the keratin protein composition exhibits a peak average molecular weight (Mp) of at least about 1.2MDa. According to one embodiment, the alpha keratin of the keratin protein composition exhibits a peak average molecular weight (Mp) of at least about 1.3MDa.

According to one embodiment, at least about 30% of the alpha keratin of the keratin protein composition exhibits a peak average molecular weight (Mp) of at least about 443 kDa. According to one embodiment, at least about 30% of the alpha keratin of the keratin protein composition exhibits a peak average molecular weight (Mp) of at least about 500 kDa. According to one embodiment, at least about 30% of the alpha keratin of the keratin protein composition exhibits a peak average molecular weight (Mp) of at least about 600 kDa. According to one embodiment, at least about 30% of the alpha keratin of the keratin protein composition exhibits a peak average molecular weight (Mp) of at least about 700 kDa. According to one embodiment, at least about 30% of the alpha keratin of the keratin protein composition exhibits a peak average molecular weight (Mp) of at least about 800 kDa. According to one embodiment, at least about 30% of the alpha keratin of the keratin protein composition exhibits a peak average molecular weight (Mp) of at least about 850 kDa. According to one embodiment, at least about 30% of the alpha keratin of the keratin protein composition exhibits a peak average molecular weight (Mp) of at least about 900 kDa. According to one embodiment, at least about 30% of the alpha keratin of the keratin protein composition exhibits a peak average molecular weight (Mp) of at least about 1.0MDa. According to one embodiment, at least about 30% of the alpha keratin of the keratin protein composition exhibits a peak average molecular weight (Mp) of at least about 1.1MDa. According to one embodiment, at least about 30% of the alpha keratin of the keratin protein composition exhibits a peak average molecular weight (Mp) of at least about 1.2MDa. According to one embodiment, at least about 30% of the alpha keratin of the keratin protein composition exhibits a peak average molecular weight (Mp) of at least about 1.3MDa.

According to one embodiment, the gamma keratin exhibits a peak average molecular weight (Mp) of from about 15 kDa to about 45 kDa. According to one embodiment, the gamma keratin exhibits a peak average molecular weight (Mp) of from about 25 kDa to about 35 kDa. According to one embodiment, the gamma keratin exhibits a peak average molecular weight (Mp) of about 30 kDa.

According to one embodiment, the hair treatment composition further includes at least one peptide that includes at least one amino acid linked by chemical bonds. The peptide aids in regenerating the scalp, nourishing the scalp, stimulating hair follicles, and creating a healthy environment for promoting existing and new hair. According to one embodiment, the at least one peptide includes at least one biomimetic signal peptide (also referred to as a biomimetic peptide). According to one embodiment, the at least one biomimetic signal peptide is acetyl tetrapeptide-3. According to one embodiment, the at least one biomimetic signal peptide is acetyl tetrapeptide-3, biotinoyl tripeptide-1, tripeptide-1, acetyl hexapeptide-8, acetyl dipeptide-1, caproyl tetrapeptide-3, carnosine, glutathione, palmitoyl oligopeptide, palmitoyl tetrapeptide-7, acetyl tetrapeptide-5, palmitoyl hexapeptide-14, pentapeptide-3, nonapeptide-1, acetyl hexapeptide, hexapeptide-11, hexanoyl dipeptide-3, acetyl octapeptide-3, palmitoyl tripeptide-5, palmitoyl dipeptide-5, palmitoyl dipeptide-6, acetyl tetrapetide-2, myristoyl pentapeptide-17, or any combination thereof.

According to one embodiment, the hair treatment compositions provided herein include about 10% w/w or less of the at least one peptide based on the total weight percent of the hair treatment composition. According to one embodiment, the hair treatment compositions provided herein include about 9% w/w or less of the at least one peptide based on the total weight percent of the hair treatment composition. According to one embodiment, the hair treatment compositions provided herein include about 8% w/w or less of the at least one peptide based on the total weight percent of the hair treatment composition. According to one embodiment, the hair treatment compositions provided herein include about 7% w/w or less of the at least one peptide based on the total weight percent of the hair treatment composition. According to one embodiment, the hair treatment compositions provided herein include about 6% w/w or less of the at least one peptide based on the total weight percent of the hair treatment composition. According to one embodiment, the hair treatment compositions provided herein include about 5% w/w or less of the at least one peptide based on the total weight percent of the hair treatment composition. According to one embodiment, the hair treatment compositions provided herein include about 4% w/w or less of the at least one peptide based on the total weight percent of the hair treatment composition. According to one embodiment, the hair treatment compositions provided herein include about 3% w/w or less of the at least one peptide based on the total weight percent of the hair treatment composition. According to one embodiment, the hair treatment compositions provided herein include about 2% w/w or less of the at least one peptide based on the total weight percent of the hair treatment composition. According to one embodiment, the hair treatment compositions provided herein include about 1% w/w or less of the at least one peptide based on the total weight percent of the hair treatment composition. According to one embodiment, the hair treatment compositions provided herein include about 0.1% w/w or less of the at least one peptide based on the total weight percent of the hair treatment composition. According to one embodiment, the hair treatment compositions provided herein include from about 0.1% w/w to about 10% w/w the at least one peptide based on the total weight percent of the hair treatment composition. According to one embodiment, the hair treatment compositions provided herein include from about 0.1% w/w to about 7% w/w the at least one peptide based on the total weight percent of the hair treatment composition. According to one embodiment, the hair treatment compositions provided herein include from about 1% w/w to about 7% w/w the at least one peptide based on the total weight percent of the hair treatment composition. According to one embodiment, the hair treatment compositions provided herein include from about 3% w/w to about 7% w/w the at least one peptide based on the total weight percent of the hair treatment composition. According to one embodiment, the hair treatment compositions provided herein include about 5% w/w the at least one peptide based on the total weight percent of the hair treatment composition.

According to one embodiment, the hair treatment composition includes at least one clover extract. According to one embodiment, the clover extract is a red clover flower extract (Trifolium pratense L.). According to one embodiment, the hair treatment compositions provided herein include about 10% w/w or less of the at least one clover extract based on the total weight percent of the hair treatment composition. According to one embodiment, the hair treatment compositions provided herein include about 9% w/w or less of the at least one clover extract based on the total weight percent of the hair treatment composition. According to one embodiment, the hair treatment compositions provided herein include about 8% w/w or less of the at least one clover extract based on the total weight percent of the hair treatment composition. According to one embodiment, the hair treatment compositions provided herein include about 7% w/w or less of the at least one clover extract based on the total weight percent of the hair treatment composition. According to one embodiment, the hair treatment compositions provided herein include about 6% w/w or less of the at least one clover extract based on the total weight percent of the hair treatment composition. According to one embodiment, the hair treatment compositions provided herein include about 5% w/w or less of the at least one clover extract based on the total weight percent of the hair treatment composition. According to one embodiment, the hair treatment compositions provided herein include about 4% w/w or less of the at least one clover extract based on the total weight percent of the hair treatment composition. According to one embodiment, the hair treatment compositions provided herein include about 3% w/w or less of the at least one clover extract based on the total weight percent of the hair treatment composition. According to one embodiment, the hair treatment compositions provided herein include about 2% w/w or less of the at least one clover extract based on the total weight percent of the hair treatment composition. According to one embodiment, the hair treatment compositions provided herein include about 1% w/w or less of the at least one clover extract based on the total weight percent of the hair treatment composition. According to one embodiment, the hair treatment compositions provided herein include about 0.1% w/w or less of the at least one clover extract based on the total weight percent of the hair treatment composition. According to one embodiment, the hair treatment compositions provided herein include from about 0.1% w/w to about 10% w/w the at least one clover extract based on the total weight percent of the hair treatment composition. According to one embodiment, the hair treatment compositions provided herein include from about 1% w/w to about 7% w/w the at least one clover extract based on the total weight percent of the hair treatment composition.

According to one embodiment, the hair treatment composition includes at least one probiotic. According to one embodiment, the hair treatment composition includes at least one probiotic yeast extract. According to one embodiment, the hair treatment composition includes at least one probiotic such as Saccharomyces lysate extract. The probiotic aids to nourish the scalp, increase scalp and hair follicle oxygenation (oxygen consumption) and help reduce irritation in/on the scalp. According to one embodiment, the hair treatment compositions provided herein include about 10% w/w or less of the at least one probiotic based on the total weight percent of the hair treatment composition. According to one embodiment, the hair treatment compositions provided herein include about 7% w/w or less of the at least one probiotic based on the total weight percent of the hair treatment composition. According to one embodiment, the hair treatment compositions provided herein include about 5% w/w or less of the at least one probiotic based on the total weight percent of the hair treatment composition. According to one embodiment, the hair treatment compositions provided herein include about 4% w/w or less of the at least one probiotic based on the total weight percent of the hair treatment composition. According to one embodiment, the hair treatment compositions provided herein include about 3% w/w or less of the at least one probiotic based on the total weight percent of the hair treatment composition. According to one embodiment, the hair treatment compositions provided herein include about 2% w/w or less of the at least one probiotic based on the total weight percent of the hair treatment composition. According to one embodiment, the hair treatment compositions provided herein include about 1% w/w or less of the at least one probiotic based on the total weight percent of the hair treatment composition. According to one embodiment, the hair treatment compositions provided herein include about 0.1% w/w or less of the at least one probiotic based on the total weight percent of the hair treatment composition. According to one embodiment, the hair treatment compositions provided herein include from about 0.1% w/w to about 10% w/w of the at least one probiotic based on the total weight percent of the hair treatment composition. According to one embodiment, the hair treatment compositions provided herein include from about 1% w/w to about 7% w/w of the at least one probiotic based on the total weight percent of the hair treatment composition. According to one embodiment, the hair treatment compositions provided herein include from about 3% w/w to about 7% w/w of the at least one probiotic based on the total weight percent of the hair treatment composition. According to one embodiment, the hair treatment compositions provided herein include about 5% w/w of the at least one probiotic based on the total weight percent of the hair treatment composition.

According to one embodiment, the hair treatment composition includes at least one active green tea or green tea extract. According to one embodiment, the hair treatment compositions provided herein include about 5% w/w or less of the at least one green tea extract based on the total weight percent of the hair treatment composition. According to one embodiment, the hair treatment compositions provided herein include about 4% w/w or less of the at least one green tea extract based on the total weight percent of the hair treatment composition. According to one embodiment, the hair treatment compositions provided herein include about 3% w/w or less of the at least one green tea extract based on the total weight percent of the hair treatment composition. According to one embodiment, the hair treatment compositions provided herein include about 2% w/w or less of the at least one green tea extract based on the total weight percent of the hair treatment composition. According to one embodiment, the hair treatment compositions provided herein include about 1% w/w or less of the at least one green tea extract based on the total weight percent of the hair treatment composition. According to one embodiment, the hair treatment compositions provided herein include about 0.1% w/w or less of the at least one green tea extract based on the total weight percent of the hair treatment composition. According to one embodiment, the hair treatment compositions provided herein include from about 1% w/w to about 5% of the at least one green tea extract based on the total weight percent of the hair treatment composition.

According to one embodiment, the hair treatment composition includes at least one rice-based humectant to provide lightweight hydration to the hair while also plumping and smoothing the hair shaft without weighing hair down. According to one embodiment, the hair treatment compositions provided herein include about 5% w/w or less of the at least one rice-based humectant based on the total weight percent of the hair treatment composition. According to one embodiment, the hair treatment compositions provided herein include about 4% w/w or less of the at least one rice-based humectant based on the total weight percent of the hair treatment composition. According to one embodiment, the hair treatment compositions provided herein include about 3% w/w or less of the at least one rice-based humectant based on the total weight percent of the hair treatment composition. According to one embodiment, the hair treatment compositions provided herein include about 2% w/w or less of the at least one rice-based humectant based on the total weight percent of the hair treatment composition. According to one embodiment, the hair treatment compositions provided herein include about 1% w/w or less of the at least one rice-based humectant based on the total weight percent of the hair treatment composition. According to one embodiment, the hair treatment compositions provided herein include about 0.1% w/w or less of the at least one rice-based humectant based on the total weight percent of the hair treatment composition. According to one embodiment, the hair treatment compositions provided herein include from about 0.1% w/w to about 10% of the at least one rice-based humectant based on the total weight percent of the hair treatment composition. According to one embodiment, the hair treatment compositions provided herein include from about 0.1% w/w to about 5% of the at least one rice-based humectant based on the total weight percent of the hair treatment composition.

According to one embodiment, the hair treatment composition includes sodium hyaluronate. The sodium hyaluronate aids in providing a sheer moisture shield to aid in thickening hair and providing a supple feel to the hair. According to one embodiment, the hair treatment compositions provided herein include about 5% w/w or less of the sodium hyaluronate on the total weight percent of the hair treatment composition. According to one embodiment, the hair treatment compositions provided herein include about 4% w/w or less of the sodium hyaluronate on the total weight percent of the hair treatment composition. According to one embodiment, the hair treatment compositions provided herein include about 3% w/w or less of the sodium hyaluronate on the total weight percent of the hair treatment composition. According to one embodiment, the hair treatment compositions provided herein include about 2% w/w or less of the sodium hyaluronate on the total weight percent of the hair treatment composition. According to one embodiment, the hair treatment compositions provided herein include about 1% w/w or less of the sodium hyaluronate on the total weight percent of the hair treatment composition. According to one embodiment, the hair treatment compositions provided herein include about 0.1% w/w or less of the sodium hyaluronate on the total weight percent of the hair treatment composition. According to one embodiment, the hair treatment compositions provided herein include about 0.1% w/w to about 10% of the sodium hyaluronate on the total weight percent of the hair treatment composition. According to one embodiment, the hair treatment compositions provided herein include about 0.1% w/w to about 5% of the sodium honate on the total weight percent of the hair treatment composition.

According to one embodiment, the hair treatment composition includes red algae extract. The red algae extract aids in providing a sheer moisture shield to aid in thickening hair and providing a supple feel to the hair. According to one embodiment, the hair treatment compositions provided herein include about 10% w/w or less of the red algae extract on the total weight percent of the hair treatment composition. According to one embodiment, the hair treatment compositions provided herein include about 7% w/w or less of the red algae extract on the total weight percent of the hair treatment composition. According to one embodiment, the hair treatment compositions provided herein include about 5% w/w or less of the red algae extract on the total weight percent of the hair treatment composition. According to one embodiment, the hair treatment compositions provided herein include about 4% w/w or less of the red algae extract on the total weight percent of the hair treatment composition. According to one embodiment, the hair treatment compositions provided herein include about 3% w/w or less of the red algae extract on the total weight percent of the hair treatment composition. According to one embodiment, the hair treatment compositions provided herein include about 2% w/w or less of the red algae extract on the total weight percent of the hair treatment composition. According to one embodiment, the hair treatment compositions provided herein include about 1% w/w or less of the red algae extract on the total weight percent of the hair treatment composition. According to one embodiment, the hair treatment compositions provided herein include about 0.1% w/w or less of the red algae extract on the total weight percent of the hair treatment composition. According to one embodiment, the hair treatment compositions provided herein include from about 0.1% w/w to about 10% w/w of the red algae extract on the total weight percent of the hair treatment composition. According to one embodiment, the hair treatment compositions provided herein include from about 3% w/w to about 7% w/w of the red algae extract on the total weight percent of the hair treatment composition.

According to one embodiment, the hair treatment composition includes microalgae extract. The microalgae adds volume and strength to hair thereby improving hair appearance and revitalization. According to one embodiment, the hair treatment compositions provided herein include about 5% w/w or less of the microalgae extract on the total weight percent of the hair treatment composition. According to one embodiment, the hair treatment compositions provided herein include about 4% w/w or less of the microalgae extract on the total weight percent of the hair treatment composition. According to one embodiment, the hair treatment compositions provided herein include about 3% w/w or less of the microalgae extract on the total weight percent of the hair treatment composition. According to one embodiment, the hair treatment compositions provided herein include about 2% w/w or less of the microalgae extract on the total weight percent of the hair treatment composition. According to one embodiment, the hair treatment compositions provided herein include about 1% w/w or less of the microalgae extract on the total weight percent of the hair treatment composition. According to one embodiment, the hair treatment compositions provided herein include about 0.1% w/w or less of the microalgae extract on the total weight percent of the hair treatment composition. According to one embodiment, the hair treatment compositions provided herein include about 0.1% w/w to about 10% w/w of the microalgae extract on the total weight percent of the hair treatment composition. According to one embodiment, the hair treatment compositions provided herein include about 0.1% w/w to about 7% w/w of the microalgae extract on the total weight percent of the hair treatment composition. According to one embodiment, the hair treatment compositions provided herein include about 3% w/w to about 7% w/w of the microalgae extract on the total weight percent of the hair treatment composition.

According to one embodiment, the hair treatment composition includes panthenol (pro-vitamin B5). The panthenol conditions hair and provides heat protection to preserve hair strength and thickness. According to one embodiment, the hair treatment compositions provided herein include about 5% w/w or less panthenol based on the total weight percent of the hair treatment composition. According to one embodiment, the hair treatment compositions provided herein include about 4% w/w or less panthenol based on the total weight percent of the hair treatment composition. According to one embodiment, the hair treatment compositions provided herein include about 3% w/w or less panthenol based on the total weight percent of the hair treatment composition. According to one embodiment, the hair treatment compositions provided herein include about 2% w/w or less panthenol based on the total weight percent of the hair treatment composition. According to one embodiment, the hair treatment compositions provided herein include about 1% w/w or less panthenol based on the total weight percent of the hair treatment composition. According to one embodiment, the hair treatment compositions provided herein include about 0.1% w/w or less panthenol based on the total weight percent of the hair treatment composition.

According to one embodiment, the hair treatment composition includes at least one active plant extract. According to one embodiment, the hair treatment composition includes larix/larch europaea wood extract. The plant extract such as the larix/larch europaea wood extract encourages the initiation of the anagen hair phase. According to one embodiment, the hair treatment composition includes Cyperus plant root oil to enhance hair softness, shine and combability without adding weight. According to one embodiment, the hair treatment compositions provided herein include about 10% w/w or less of the at least one active plant extract based on the total weight percent of the hair treatment composition. According to one embodiment, the hair treatment compositions provided herein include about 7% w/w or less of the at least one active plant extract based on the total weight percent of the hair treatment composition. According to one embodiment, the hair treatment compositions provided herein include about 5% w/w or less of the at least one active plant extract based on the total weight percent of the hair treatment composition. According to one embodiment, the hair treatment compositions provided herein include about 4% w/w or less of the at least one active plant extract based on the total weight percent of the hair treatment composition. According to one embodiment, the hair treatment compositions provided herein include about 3% w/w or less of the at least one active plant extract based on the total weight percent of the hair treatment composition. According to one embodiment, the hair treatment compositions provided herein include about 2% w/w or less of the at least one active plant extract based on the total weight percent of the hair treatment composition. According to one embodiment, the hair treatment compositions provided herein include about 1% w/w or less of the at least one active plant extract based on the total weight percent of the hair treatment composition. According to one embodiment, the hair treatment compositions provided herein include about 0.1% w/w or less of the at least one active plant extract based on the total weight percent of the hair treatment composition. According to one embodiment, the hair treatment compositions provided herein include from about 0.1% w/w to about 10% w/w of the at least one active plant extract based on the total weight percent of the hair treatment composition. According to one embodiment, the hair treatment compositions provided herein include from about 1% w/w to about 7% w/w of the at least one active plant extract based on the total weight percent of the hair treatment composition. According to one embodiment, the hair treatment compositions provided herein include from about 3% w/w to about 7% w/w of the at least one active plant extract based on the total weight percent of the hair treatment composition.

According to one embodiment, the hair treatment composition is free of active pharmaceutical ingredients. According to one embodiment, the hair treatment composition includes at least one active pharmaceutical ingredient for supporting hair growth. According to one embodiment, the at least one active pharmaceutical ingredient for supporting hair growth is minoxidil. According to one embodiment, the minoxidil supports new hair growth. According to one embodiment, the minoxidil supports existing hair growth. According to one embodiment, the hair treatment compositions provided herein include about 15% w/w or less minoxidil based on the total weight percent of the hair treatment composition. According to one embodiment, the hair treatment compositions provided herein include about 10% w/w or less minoxidil based on the total weight percent of the hair treatment composition. According to one embodiment, the hair treatment compositions provided herein include about 5% w/w or less minoxidil based on the total weight percent of the hair treatment composition.

According to one embodiment, the hair treatment composition further includes a base solution or carrier fluid. The base solution may include one more ingredients that do not impact or change the shape of conformation of the keratin protein. Such ingredients may include one or more naturally occurring components. The base solution may include one or more of pink pomelo, hydrolyzed quinoa, artichoke leaf, vitamin A, vitamin C, vitamin B1, zinc, gotu kola, tapioca starch, kaolin clay, pea protein, phospholipids, brown algae, silica silylate, hydrated silica, asiaticoside/madecassoside, panthenol, cypress, baobab seed oil, or any combination thereof. The base solution may also include one or more of tocopherol, dimethicone, parabens, titanium dioxide, sodium lauryl sulphate, sodium laureth sulphate, retinol, collagen, ambergris, squalene, cochineal dye, guanine, tallow, gelatin, lanolin, citric acid, sodium citrate, cocamide, guar gum, xanthum gujm, glycol distearate, polyglycol esters, sodium chloride, glycerin, cetyl alcohol, stearyl alcohol, panthenol, silicones, or any combination thereof. The base solution may also include one or more of D-panthenol, polysorbate 20, cetyl alcohol, Crambe abyssinica seed oil, hydroxyethyl acrylate/sodium acryloyldimethyl taurate copolymer, isohexadecane, polysorbate 60, cyclopentasiloxane, cyclohexasiloxane, C11-13 isoparaffin, dimethiconol, isohexadecane, dimethicone, caprylyl glycol, phenoxyethanol, hexylene glycol, or any combination thereof. The base solution may include on or more of the additives, polymers, solvents, or film forming agents as described herein.

According to one embodiment, the hair treatment composition includes at least one preservative. Suitable preservatives include, but are not limited to, any preservative that is acceptable for hair treatment purposes.

According to one embodiment, the hair treatment composition includes water. According to a particular embodiment, the water is sterile. The water is present in an amount of from about 0.01% w/w to about 99.99% w/w based on the total weight of the hair treatment composition. According to one embodiment, the hair treatment composition includes at least about 50% w/w water based on the total weight of the hair treatment composition. According to one embodiment, the hair treatment composition includes at least about 60% w/w water based on the total weight of the hair treatment composition. According to one embodiment, the hair treatment composition includes at least about 70% w/w water based on the total weight of the hair treatment composition. According to one embodiment, the hair treatment composition includes at least about 80% w/w water based on the total weight of the hair treatment composition. According to one embodiment, the hair treatment composition includes at least about 90% w/w water based on the total weight of the hair treatment composition. According to one embodiment, the hair treatment composition includes about 50% w/w or less water based on the total weight of the hair treatment composition. According to one embodiment, the hair treatment composition includes about 40% w/w or less water based on the total weight of the hair treatment composition. According to one embodiment, the hair treatment composition includes about 30% w/w or less water based on the total weight of the hair treatment composition. According to one embodiment, the hair treatment composition includes about 20% w/w or less water based on the total weight of the hair treatment composition. According to one embodiment, the hair treatment composition includes about 10% w/w or less water based on the total weight of the hair treatment composition. According to one embodiment, the hair treatment composition includes about 5% w/w or less water based on the total weight of the hair treatment composition. According to one embodiment, the hair treatment composition includes about 1% w/w or less water based on the total weight of the hair treatment composition.

According to one embodiment, the hair treatment composition further includes one or more hair treatment additives. The hair treatment additives do not themselves change the functional properties of any keratin protein present in the hair treatment composition. Suitable hair treatment additives include, but are not limited to, non-ionic, anionic or cationic polymers, preservatives, oils, pH regulators, waxes, fragrances, antifatting agents, sequestrating agents, perfumes, cationic surfactants, proteins, silicones, surfactants, organic solvents and any other additive conventionally used in the hair treatments field. According to one embodiment, the at least one hair treatment additive is present in an amount of between about 0.01% w/w and about 80% w/w based on the total weight of the hair treatment composition. According to one embodiment, the at least one hair treatment additive is present in an amount of about 80% w/w or less based on the total weight of the hair treatment composition. According to one embodiment, the at least one hair treatment additive is present in an amount of about 70% w/w or less based on the total weight of the hair treatment composition. According to one embodiment, the at least one hair treatment additive is present in an amount of about 60% w/w or less based on the total weight of the hair treatment composition. According to one embodiment, the at least one hair treatment additive is present in an amount of about 50% w/w or less based on the total weight of the hair treatment composition. According to one embodiment, the at least one hair treatment additive is present in an amount of about 40% w/w or less based on the total weight of the hair treatment composition. According to one embodiment, the at least one hair treatment additive is present in an amount of about 30% w/w or less based on the total weight of the hair treatment composition. According to one embodiment, the at least one hair treatment additive is present in an amount of about 20% w/w or less based on the total weight of the hair treatment composition. According to one embodiment, the at least one hair treatment additive is present in an amount of about 10% w/w or less based on the total weight of the hair treatment composition.

According to one embodiment, the hair treatment composition includes one or more polymers to provide hold to the hair. Suitable polymers include, but are not limited to, nonionic polymers such as polyvinylpyrrolidones, copolymers of polyvinylpyrrolidone and vinyl acetate, and anionic polymers such as copolymers of vinyl acetate and an unsaturated carboxylic acid such as crotonic acid, copolymers resulting from copolymerisation of vinyl acetate, crotonic acid and an acrylic or methacrylic ester, copolymers resulting from copolymerisation of vinyl acetate, an alkoylvinyl ether and an unsaturated carboxylic acid and copolymers resulting from copolymerisation of vinyl acetate, crotonic acid and a vinyl ester of a long carbon chain acid or an allyl or methallyl ester of a long carbon chain acid. According to one embodiment, the at least one polymer is present in an amount of between about 0.01% w/w and about 80% w/w based on the total weight of the hair treatment composition. According to one embodiment, the at least one polymer is present in an amount of about 80% w/w or less based on the total weight of the hair treatment composition. According to one embodiment, the at least one polymer is present in an amount of about 70% w/w or less based on the total weight of the hair treatment composition. According to one embodiment, the at least one polymer is present in an amount of about 60% w/w or less based on the total weight of the hair treatment composition. According to one embodiment, the at least one polymer is present in an amount of about 50% w/w or less based on the total weight of the hair treatment composition. According to one embodiment, the at least one polymer is present in an amount of about 40% w/w or less based on the total weight of the hair treatment composition. According to one embodiment, the at least one polymer is present in an amount of about 30% w/w or less based on the total weight of the hair treatment composition. According to one embodiment, the at least one polymer is present in an amount of about 20% w/w or less based on the total weight of the hair treatment composition. According to one embodiment, the at least one polymer is present in an amount of about 10% w/w or less based on the total weight of the hair treatment composition. According to one embodiment, the at least one polymer is present in an amount of about 5% w/w or less based on the total weight of the hair treatment composition. According to one embodiment, the at least one polymer is present in an amount of about 1% w/w or less based on the total weight of the hair treatment composition.

According to one embodiment, the hair treatment composition includes one or more solvents acceptable in hair treatment compositions. Suitable acceptable solvents include low alcohols, for example ethanol or isopropanol, glycerol, glycols or glycol ethers such as the monobutyl ether of ethyleneglycol, propyleneglycol, or the monoethylether or the monomethylether of diethyleneglycol, in proportions which do not affect gel formation. According to one embodiment, the at least one solvent is present in an amount of between about 0.01% w/w and about 99.9% w/w based on the total weight of the hair treatment composition. According to one embodiment, the at least one solvent is present in an amount of about 90% w/w or less based on the total weight of the hair treatment composition. According to one embodiment, the at least one solvent is present in an amount of about 80% w/w or less based on the total weight of the hair treatment composition. According to one embodiment, the at least one solvent is present in an amount of about 70% w/w or less based on the total weight of the hair treatment composition. According to one embodiment, the at least one solvent is present in an amount of about 60% w/w or less based on the total weight of the hair treatment composition. According to one embodiment, the at least one solvent is present in an amount of about 50% w/w or less based on the total weight of the hair treatment composition. According to one embodiment, the at least one solvent is present in an amount of about 40% w/w or less based on the total weight of the hair treatment composition. According to one embodiment, the at least one solvent is present in an amount of about 30% w/w or less based on the total weight of the hair treatment composition. According to one embodiment, the at least one solvent is present in an amount of about 20% w/w or less based on the total weight of the hair treatment composition. According to one embodiment, the at least one solvent is present in an amount of about 10% w/w or less based on the total weight of the hair treatment composition. According to one embodiment, the at least one solvent is present in an amount of about 1% w/w or less based on the total weight of the hair treatment composition.

According to one embodiment, the hair treatment compositions provided herein include less than about 5% w/w of the methyl nicotinate based on the total weight percent of the hair treatment composition. According to one embodiment, the hair treatment compositions provided herein include less than about 3% w/w of the methyl nicotinate based on the total weight percent of the hair treatment composition. According to one embodiment, the hair treatment compositions provided herein include less than about 1% w/w of the methyl nicotinate based on the total weight percent of the hair treatment composition. According to one embodiment, the hair treatment compositions provided herein include less than about 0.1% w/w of the methyl nicotinate based on the total weight percent of the hair treatment composition. According to one embodiment, the hair treatment compositions provided herein include between about 0.1% w/w and about 5% of the methyl nicotinate based on the total weight percent of the hair treatment composition. According to one embodiment, the hair treatment compositions provided herein include between about 0.5% w/w and about 3% of the methyl nicotinate based on the total weight percent of the hair treatment composition.

According to one embodiment, the hair treatment composition may further include at least one or more additional additives. Suitable additional additives include, but are not limited to, solid emollients, emulsifiers, surface active agent, gum, humectant, thickener, powder diluent, dispersant, or carrier so as to facilitate the distribution of the composition when applied. According to one embodiment, the at least one additional additive is present in an amount of between about 0.1% w/w and about 99.9% w/w based on the total weight of the hair treatment composition.

According to one embodiment, the hair treatment composition may further include a film forming agent. Suitable firm forming agents include, but are not limited to, liquid or solid emollient, emulsifiers, surface active agents, gums, humectants, thickeners, powders and protein solutions. Suitable emollients include mineral oil, fatty alcohols, alkyl esters, silicones and silicone derivatives. According to one embodiment, the at least one film forming agent is present in an amount of between about 0.01% w/w and about 99.9% w/w based on the total weight of the hair treatment composition.

According to one embodiment, the hair treatment composition includes a keratin protein composition, water, and one or more of cetearyl alcohol, dimethicone, brassicamidopropyl dimethylamine, cetyl esters, cetyl alcohol, behentrimonium chloride, isododecane, hydroxyethylcellulose, polysorbate 60, benzyl alcohol, sorbic acid, benzoic acid, glycerin, phenoxyethanol, dimethiconol, chlorphenesin, gluconolactone, ethylhexylglycerin, PGE-100 stearate, glyceryl stearate, Silybum marianum, ethyl ester, tocopheryl nicotinate, Cyperus esculentus root oil, madecassoside, Quercus Patrea fruit extract, hydrolyzed quinoa, Oryza sativa (rice) extract, Rosmarinus Officinalis (Rosemary) leaf extract, hydrogenated ethylhexyl olivate, Oryza sativa (rice) bran extract, hydrogenated olive oil unsaponifiables, jojoba esters, Helianthus Annuus (sunflower) seed oil, Oryza Sativa (rice) seed protein, phytic acid, tetrasodium glutamate diacetate, methyl nicotinate, acetyl tetrapeptide-3, sodium lauroyl methyl isethionate, sodium cocoyl isethionate, lauryl glucoside, acrylates copolymer, sodium hyaluronate, rice oil, glycereth-8 esters, cocamide MIPA, polysorbate 20, hydrogenated coconut acid, sodium isethionate, sodium methyl cocoyl taurate, caprylyl glycol, guar hydroxypropyltrimonium chloride, Chondrus crispus extract, ethylhexyl salicylate, sodium hydroxide, sodium chloride, Zingiber Officinale (ginger) root extract, Pisum Sativum (Pea) extract, Nasturtium Officinale extract, Moringa Oleifera seed extract, Jasminum Officinale (Jasmine) flower extract, hydrolyzed linseed extract, hydrolyzed Hibiscus Esculentus extract, Helianthus Annuus (sunflower) seed extract, Daucus Carota Sativa (Carrot) root extract, Cynara scolymus (artichoke) leaf extract, Amaranthus caudatus seed extract, Saccharomyces Lysate extract (probiotic yeast for increasing oxygenation and reducing irritation in the scalp), Larix europaea wood extract, Trifolium pratense (clover) flower extract, Camellia sinensis leaf extract, Isochrysis galbana extract, tocopherol, Mentha piperita (Peppermint) oil, pentylene glycol, sodium phytate, sodium dilauramidoglutamide lysine, quaternium-51, ethylhexylglycerin, sodium metabisulfite, glycine, a fragrance, biosaccharide gum-4, butylene glycol, glycerin, zinc chloride, PPG-26-Buteth-26, propanediol, polyacrylate crosspolymer-6, PEG-40 hydrogenated castor oil, t-butyl alcohol, alcohol, sodium cocoyl glutamate, tetrasodium EDTA, trisodium ethylenediamine disuccinate, butylene glycol, polyglyceryl-6 oleate, glyceryl caprylate, sodium dilauramidoglutamide lysine, caprylyl/capryl glucoside, ethylhexylglycerin, sodium benzoate, potassium sorbate, disodium EDTA, phenoxyethanol, sodium surfactin, dextran, citric acid, linalool, geraniol, limonene, citronellol, benzyl benzoate, citral, hexyl cinnamal, benzophenone-4, benzophenoneo-3, or any combination thereof.

According to one embodiment, the hair treatment composition as provided herein is free of sulfates, parabens phthalates, synthetic colors, dyes and gluten.

According to one embodiment, the hair treatment composition includes a complex formed from: a keratin protein composition that includes alpha keratin and gamma keratin; at least one biomimetic signal peptide; and red clover extract. According to one embodiment, the hair treatment composition includes a complex formed from: a keratin protein composition that consists of alpha keratin and gamma keratin; at least one biomimetic signal peptide; and red clover extract. According to one embodiment, the hair treatment composition includes: a keratin protein composition that includes alpha keratin and gamma keratin; at least one biomimetic signal peptide; and red clover extract. According to one embodiment, the hair treatment composition includes a complex that consists of: a keratin protein composition that includes or consists of alpha keratin and gamma keratin; at least one biomimetic signal peptide; and red clover extract.

According to one embodiment, the hair treatment composition may be formulated to be applied to any part of the body where a hair treatment application is acceptable. According to one embodiment, the hair treatment composition may be formulated to be applied to hair, scalp or a combination thereof. According to one embodiment, the hair treatment composition may be formulated as an shampoo, conditioner, aqueous solution, powder, gel, mousse/foam, spray, dressing, lotion, wax, hydrogel, oil, emulsion, paste, polish or cream. According to one embodiment, the hair treatment compositions as provided herein may be formulated as hair care products such as shampoo, conditioner, spray, or foam. According to one embodiment, the hair treatment compositions as provided herein may be formulated as or incorporated within a moisturizer, a deodorant, an anti-aging/skin repair preparation, a cleanser, a toner, an eye care composition, a lip care composition, a sun care composition (e.g., sunscreen), a hand care composition, or a body care composition.

According to one embodiment, the hair treatment composition is a shampoo that includes a keratin protein composition as provided herein, water, sodium hyaluronate, red algae extract, rice-based humectant (e.g., rice oil glycereth-8 esters), and at least one biomimetic signal peptide such as acetyl tetrapeptide-3 with each of the aforementioned components present in the amounts provided herein. According to one embodiment, the hair treatment composition that is a shampoo further includes one or more additional components including Sodium Lauroyl Methyl Isethionate, Sodium Cocoyl Isethionate, Lauryl Glucoside, Acrylates Copolymer, Trifolium pratense (Clover) Flower Extract, Camellia sinensis Leaf Extract, Larix europaea Wood Extract, Tocopherol, Sodium Phytate, Cocamide MIPA, Polysorbate 20, Hydrogenated Coconut Acid, Sodium Isethionate, Sodium Methyl Cocoyl Taurate, Caprylyl Glycol, Guar Hydroxypropyltrimonium Chloride, Trisodium Ethylenediamine Disuccinate, Ethylhexylglycerin, Hexylene Glycol, Glycerin, Butylene Glycol, Sodium Dilauramidoglutamide Lysine, Isopropanolamine, Coconut Acid, Dextran, Chondrus crispus Extract, Ethylhexyl Salicylate, Sodium Metabisulfite, Glycine, Gluconolactone, Zinc Chloride, Phenoxyethanol, Parfum (Fragrance), Potassium Sorbate, Sodium Hydroxide, Citric Acid, Sodium Benzoate, Sodium Chloride, Linalool, Geraniol, Limonene, and Citronellol, with the aforementioned additional components present in an amount of 70% w/w or less based on the total weight of the hair treatment composition.

According to one embodiment, the hair treatment composition is a conditioner that includes a keratin protein composition as provided herein, water, red cover extract, at least one biomimetic signal peptide such as acetyl tetrapeptide-3, Cyperus esculentus root oil, and at least one probiotic such as Saccharomyces lysate extract, with each of the aforementioned components present in the amounts provided herein. According to one embodiment, the hair treatment composition that is a conditioner further includes one or more additional components including Cetearyl Alcohol, Dimethicone, Brassicamidopropyl Dimethylamine, Cetyl Esters, Cetyl Alcohol, Behentrimonium Chloride, Isododecane, Ethylhexylglycerin, PEG-100 Stearate, Glyceryl Stearate, Silybum marianum Ethyl Ester, Panthenol, Tocopheryl Nicotinate, Tocopherol, Madecassoside, Quercus petraea Fruit Extract, Citrus paradisi (Grapefruit) Fruit Extract, Asiaticoside, Hydrolyzed Adansonia digitata Seed Extract, Amaranthus hypochondriacus Leaf Extract, Trifolium pratense (Clover) Flower Extract, Larix europaea Wood Extract, Glycine, Hydrolyzed Vegetable Protein, Hydrolyzed Quinoa, Oryza sativa (Rice) Extract, Hydrolyzed Linseed Extract, Camellia sinensis Leaf Extract, Rosmarinus officinalis (Rosemary) Leaf Extract, Hydrogenated Ethylhexyl Olivate, Oryza sativa (Rice) Bran Extract, Hydrogenated Olive Oil Unsaponifiables, Jojoba Esters, Helianthus annuus (Sunflower) Seed Oil, Oryza sativa (Rice) Seed Protein, Phytic Acid, Tetrasodium Glutamate Diacetate, Butylene Glycol, Sodium Dilauramidoglutamide Lysine, Dextran, Disodium Phosphate, Caprylyl Glycol, Propanediol, Parfum (Fragrance), Hydroxyethylcellulose, Polysorbate 60, Benzyl Alcohol, Polysorbate 20, Sorbic Acid, Benzoic Acid, Glycerin, Phenoxyethanol, Dimethiconol, Chlorphenesin, Sodium Metabisulfite, Gluconolactone, Zinc Chloride, Sodium Hydroxide, Alcohol, Citric Acid, Tetrasodium EDTA, Sodium Benzoate, Sodium Phosphate, Potassium Sorbate, Calcium Gluconate, BHT, Linalool, Citronellol, Geraniol, and Limonene, with the aforementioned additional components present in an amount of 70% w/w or less based on the total weight of the hair treatment composition.

According to one embodiment, the hair treatment composition is a spray that includes a keratin protein composition as provided herein, water, red clover extract, at least one biomimetic signal peptide, at least one probiotic such as Saccharomyces lysate extract, larix/larch europaea wood extract, and methyl nicotinate, with each of the aforementioned components present in the amounts provided herein. According to one embodiment, the hair treatment composition that is a spray further includes one or more additional components including Zingiber officinale (Ginger) Root Extract, Pisum sativum (Pea) Extract, Nasturtium Officinale Extract, Moringa Oleifera Seed Extract, Jasminum officinale (Jasmine) Flower Extract, Hydrolyzed Linseed Extract, Hydrolyzed Hibiscus Esculentus Extract, Helianthus annuus (Sunflower) Seed Extract, Daucus carota sativa (Carrot) Root Extract, Cynara scolymus (Artichoke) Leaf Extract, Amaranthus caudatus Seed Extract, Trifolium pratense (Clover) Flower Extract, Camellia sinensis Leaf Extract, lsochrysis Galbana Extract, Tocopherol, Mentha piperita (Peppermint) Oil, Dextran, Pentylene Glycol, Sodium Phytate, Sodium Dilauramidoglutamide Lysine, Quaternium-51, Ethylhexylglycerin, Sodium Metabisulfite, Glycine, Parfum (Fragrance), Biosaccharide Gum-4, Butylene Glycol, Glycerin, Zinc Chloride, PPG-26-Buteth-26, Propanediol, Polyacrylate Crosspolymer-6, PEG-40 Hydrogenated Castor Oil, t-Butyl Alcohol, Alcohol, Tetrasodium EDTA, Sodium Benzoate, Potassium Sorbate, Disodium EDTA, Phenoxyethanol, Citric Acid, Linalool, Geraniol, Limonene, Citronellol, Benzyl Benzoate, Citral, and Hexyl Cinnamal, with the aforementioned additional components present in an amount of 80% w/w or less based on the total weight of the hair treatment composition.

According to one embodiment, the hair treatment composition is a foam that includes a keratin protein composition as provided herein, water, panthenol, plant extracts, and microalgae extract, with each of the aforementioned components present in the amounts provided herein. The foam may further include at least one at least one active pharmaceutical ingredient for supporting hair health and increasing anagen hair count or percentage. According to one embodiment, the at least one active pharmaceutical ingredient for supporting hair growth is minoxidil. The minoxidil may present in an amount of about 5% w/w based on the total weight of the foam. According to one embodiment, the hair treatment composition that is foam further includes one or more additional components including AMP-Acrylates/Allyl Methacrylate Copolymer, Biosaccharide Gum-4, lsochrysis Galbana Extract, Acetyl Tetrapeptide-3, Zingiber officinale (Ginger) Root Extract, Pisum sativum (Pea) Extract, Nasturtium Officinale Extract, Moringa Oleifera Seed Extract, Jasminum officinale (Jasmine) Flower Extract, Hydrolyzed Linseed Extract, Hydrolyzed Hibiscus Esculentus Extract, Helianthus annuus (Sunflower) Seed Extract, Daucus carota sativa (Carrot) Root Extract, Cynara scolymus (Artichoke) Leaf Extract, Amaranthus caudatus Seed Extract, Trifolium pratense (Clover) Flower Extract, Sodium Metabisulfite, Larix europaea Wood Extract, Glycine, Tocopherol, Zinc Chloride, Camellia sinensis Leaf Extract, Sodium Cocoyl Glutamate, Trisodium Ethylenediamine Disuccinate, Butylene Glycol, Polyglyceryl-6 Oleate, Glyceryl Caprylate, Sodium Dilauramidoglutamide Lysine, Caprylyl/Capryl Glucoside, Ethylhexylglycerin, Parfum (Fragrance), Glycerin, Pentylene Glycol, Sodium Surfactin, Dextran, Phenoxyethanol, Benzophenone-4, Benzophenone-3, Alcohol, Citric Acid, Linalool, Geraniol, Limonene, Citronellol, Benzyl Benzoate, Citral, and Hexyl Cinnamal, with the aforementioned additional components present in an amount of 80% w/w or less based on the total weight of the hair treatment composition.

According to one embodiment, the hair treatment compositions as provided herein are particularly suited for improving overall scalp health. The hair treatment compositions as provided herein are particularly suited for nourishing or otherwise treating, maintaining and improving the health of the scalp microbiome. The hair treatment compositions as provided herein cleanse a hair shaft and clear debris from follicle openings, creating an environment for healthy hair and an increase in anagen hair. According to one embodiment, the hair treatment compositions as provided herein include at least one vasodilator that works synergistically the remaining components in the hair treatment composition.

According to one embodiment, the hair treatment compositions as provided herein are particularly suited for maintaining a healthy scalp pH. The hair treatment compositions as provided herein are suited to raise and maintain pH at a level that remains below 5.5 but above 4.5 as well as above the pH value of hair (about 3.6). The slightly acidic environment provides a scalp environment that prevents fungal and bacterial overgrowth while keeping hair follicles relaxed/open and healthy. The hair treatment compositions as provided herein also nourish and maintain the natural acidic sebum of the scalp.

According to one embodiment, the hair treatment compositions as provided herein are particularly suited for maintaining or nourishing any beneficial microorganisms on the scalp. By improving scalp microbiome, common scalp conditions such as flaking, seborrheic dermatitis, dandruff, and psoriasis are treated or otherwise prevented. The hair treatment composition as provided herein aids in the maintenance and improvement of the scalp microbiome by aiding in the balance of the various microorganisms that typically reside on the surface of the scalp including fungi, yeast, viruses, algae and bacteria.

A hair treatment regimen kit is provided. According to one embodiment, the hair treatment regimen kit includes a hair treatment composition formulated as a shampoo as provided herein. According to one embodiment the shampoo includes a keratin protein composition as provided herein, water, red clover extract, sodium hyaluronate, red algae extract, rice oil glycereth-8 esters, and at least one biomimetic signal peptide such as acetyl tetrapeptide-3. According to one embodiment, the hair treatment regimen kit includes a hair treatment composition formulated as a conditioner as provided herein. According to one embodiment, the conditioner includes a keratin protein composition as provided herein, water, red clover extract, at least one biomimetic signal peptide such as acetyl tetrapeptide-3, Cyperus esculentus root oil, and at least one probiotic such as Saccharomyces lysate extract. According to one embodiment, the hair treatment regimen kit includes a hair treatment composition formulated as a spray as provided herein. According to one embodiment, the spray includes a keratin protein composition as provided herein, water, red clover extract, at least one biomimetic signal peptide, at least one probiotic such as Saccharomyces lysate extract, larix/larch europaea wood extract, and methyl nicotinate. According to one embodiment, the hair treatment regimen kit includes a foam that includes minoxidil. According to one embodiment, the minoxidil is present at a concentration of about 0.1% w/w to about 15% w/w. According to one embodiment, the minoxidil is present at a concentration of about 5% w/w. According to one embodiment, the hair treatment regimen kit includes a hair treatment composition formulated as a foam. According to one embodiment, the foam includes a keratin protein composition as provided herein, water, panthenol, plant extracts, and microalgae extract. The hair treatment regimen kit may be utilized by a user in need of treatment for supporting or promoting an increase in the percentage of anagen hair, increasing or otherwise promoting hair density, providing a noticeable increase in the percentage of anagen hair, decreasing thinning hair, reducing hair shedding, improving scalp health, increasing the pH of the scalp, or any combination thereof.

According to one embodiment, a method for the production of a keratin protein composition is provided. The method includes the step obtaining or providing a keratin protein source. The keratin protein source is human hair. According to one embodiment, the human hair is of Asian origin. The keratin protein source is not derived from other sources such as, for example, wool, fur, horns, hooves, beaks, feathers, scales. According to one embodiment, the method further includes the step of separating and dialyzing a liquid extract resulting in a soluble keratin protein solution. According to one embodiment, the keratin protein is alpha keratin, gamma keratin, or a combination thereof. According to one embodiment, the method further includes the step of adding one or more of the hair treatment composition components described herein.

According to one embodiment, the keratin protein as provided herein is not precipitated during production of the keratin protein composition. According to one embodiment, the keratin protein maintains a naturally occurring shape or configuration (i.e., does not undergo any transformation or denaturing) during extraction from human hair and subsequent processing. According to one embodiment, the keratin protein does not undergo any transformation or denaturing during extraction from human hair and subsequent processing. The extraction process ensures the harvested proteins are of correct conformation structure. According to one embodiment, the keratin proteins as provided herein are substantially or completely soluble in water. Still further, the keratin proteins provided herein exhibit improved performance for the treatment and repair of hair and scalp compared to compositions that include denatured keratin proteins.

### Methods of Treatment

The compositions provided herein are suitable for various hair and scalp treatment purposes on the exterior of a mammalian body. The hair treatment compositions as provided herein may be utilized to strengthen or improve the overall appearance and health of a mammal's hair. According to one embodiment, the hair treatment compositions provided herein may be utilized in an effective amount for the treatment of keratin-comprising structures and tissues. The hair treatment compositions provided herein are suitable for improving or supporting scalp health, increasing and maintaining a healthy scalp pH, maintaining and improving the health of the scalp microbiome, improving scalp flora ecology, and maintaining scalp microbiome diversity. The hair treatment compositions provided herein may be utilized in the manufacture of a medicament for the various uses or methods of treatment provided herein.

In general, the hair treatment compositions provided herein may be topically administered at least on a daily basis for a period of time sufficient to bring about the desired level of improvement in the hair, scalp, or a combination thereof. The hair treatment compositions may be alternatively applied, once, twice, three, four, five or six times a week. The hair treatment compositions may be alternatively applied be applied once daily or every other day. A user may topically administer a hair treatment composition directly to the hair and scalp by gently massaging, spraying, dabbing, swabbing, or rubbing the requisite hair treatment composition into the desired area. In some embodiments, the hair treatment composition may be temporarily left on the desired area. Topical application of the hair treatment compositions as provided herein may continue for any suitable period of time including from a few days to a few weeks to a few months or on a daily basis for as long as desired. Duration of treatment with which the hair treatment compositions may vary depending on the desired effect. In some embodiments, the degree of therapeutic and/or cosmetic enhancement will vary directly with the total amount and length of time the hair treatment composition is used.

A method for reducing or otherwise treating or preventing hair loss is provided. The method includes the steps of administering an effective amount of at least one hair treatment composition as provided herein to a surface of the hair and scalp in need of treatment thereby improving overall scalp and hair health to reduce, treat or prevent hair loss.

A method of supporting new hair growth is provided that includes the step of administering an effective amount of at least one hair treatment composition as provided herein to a surface of the hair and scalp in need of treatment. A method of supporting existing hair growth is provided that includes the step of administering an effective amount of at least one hair treatment composition as provided herein to a surface of the hair and scalp in need of treatment.

A method for the treatment or management of alopecia is provided that includes the step of administering to a subject having alopecia an effective amount of at least one hair treatment composition as provided herein. The alopecia may be attributed to or caused by hereditary factors, hormonal fluctuations, recent pregnancy, nutritional deficiencies, stress, or any combination thereof. According to one embodiment, the alopecia is androgenetic alopecia. According to one embodiment, the alopecia is androgenetic alopecia of a female human. According to one embodiment, the alopecia is androgenetic alopecia of a female human's scalp.

A method for the treatment or management of telogen effluvium is provided that includes the step of administering an effective amount of at least one hair treatment composition as provided herein to a surface of the hair and scalp in need of treatment.

A method of increasing hair thickness is provided includes the step of administering an effective amount of at least one hair treatment composition as provided herein to a surface of the hair and scalp in need of treatment. According to one embodiment, the hair treatment composition clears hair follicles and strengthens a moisture barrier on the scalp to improve hair health.

A method of decreasing hair thinning is provided that includes the step of administering an effective amount of at least one hair treatment composition as provided herein to a surface of the hair and scalp in need of treatment. According to one embodiment, the thinning hair is due to aging.

A method of decreasing hair shedding is provided that includes the step of administering an effective amount of at least one hair treatment composition as provided herein to a surface of the hair and scalp in need of treatment.

A method of increasing or otherwise promoting hair density is provided that includes the step of administering an effective amount of one or more hair treatment composition as provided herein to a surface of the hair and scalp in need of treatment. According to one embodiment, the hair treatment composition increases hair density by at least about 5%, 10%, 15%, 20%, 25%, 30%, 35%, 40%, 45%, or 50% after 120 days of daily administration. According to one embodiment, the hair treatment composition increases hair density by at least about 5%, 10%, 15%, 20%, 25%, 30%, 35%, 40%, 45%, or 50% after 180 days of daily administration. According to one embodiment, the hair treatment composition increases hair density by at least about 10% after 120 days of daily administration of the hair treatment composition.

A method of increasing or otherwise promoting hair density is provided that includes the step of administering an effective amount of one or more hair treatment composition as provided herein to a surface of the hair and scalp in need of treatment, wherein the hair treatment composition is formulated as a shampoo. According to one embodiment, the hair treatment composition increases hair density by at least about 5%, 10%, 15%, 20%, 25%, 30%, 35%, 40%, 45%, or 50% after 120 days of daily administration. According to one embodiment, the hair treatment composition increases hair density by at least about 5%, 10%, 15%, 20%, 25%, 30%, 35%, 40%, 45%, or 50% after 180 days of daily administration. According to one embodiment, the hair treatment composition increases hair density by at least about 10% after 120 days of daily administration of the hair treatment composition.

A method of increasing or otherwise promoting hair density is provided that includes the step of administering an effective amount of one or more hair treatment composition as provided herein to a surface of the hair and scalp in need of treatment, wherein the hair treatment composition is formulated as a conditioner. According to one embodiment, the hair treatment composition increases hair density by at least about 5%, 10%, 15%, 20%, 25%, 30%, 35%, 40%, 45%, or 50% after 120 days of daily administration. According to one embodiment, the hair treatment composition increases hair density by at least about 5%, 10%, 15%, 20%, 25%, 30%, 35%, 40%, 45%, or 50% after 180 days of daily administration. According to one embodiment, the hair treatment composition increases hair density by at least about 10% after 120 days of daily administration of the hair treatment composition.

A method of increasing or otherwise promoting hair density is provided that includes the step of administering an effective amount of one or more hair treatment composition as provided herein to a surface of the hair and scalp in need of treatment, wherein the hair treatment composition is formulated as a spray. According to one embodiment, the hair treatment composition increases hair density by at least about 5%, 10%, 15%, 20%, 25%, 30%, 35%, 40%, 45%, or 50% after 120 days of daily administration. According to one embodiment, the hair treatment composition increases hair density by at least about 5%, 10%, 15%, 20%, 25%, 30%, 35%, 40%, 45%, or 50% after 180 days of daily administration. According to one embodiment, the hair treatment composition increases hair density by at least about 10% after 120 days of daily administration of the hair treatment composition.

A method of increasing or otherwise promoting hair density is provided that includes the step of administering an effective amount of one or more hair treatment composition as provided herein to a surface of the hair and scalp in need of treatment, wherein the hair treatment composition is formulated as a foam. According to one embodiment, the hair treatment composition increases hair density by at least about 5%, 10%, 15%, 20%, 25%, 30%, 35%, 40%, 45%, or 50% after 120 days of daily administration. According to one embodiment, the hair treatment composition increases hair density by at least about 5%, 10%, 15%, 20%, 25%, 30%, 35%, 40%, 45%, or 50% after 180 days of daily administration. According to one embodiment, the hair treatment composition increases hair density by at least about 10% after 120 days of daily administration of the hair treatment composition.

A method of increasing or otherwise promoting hair density is provided that includes the steps of: administering an effective amount of a first hair treatment composition as provided herein to a surface of the hair and scalp in need of treatment; administering an effective amount of a second hair treatment composition as provided herein to a surface of the hair and scalp in need of treatment; and administering an effective amount of a third hair treatment composition as provided herein to a surface of the hair and scalp in need of treatment, wherein the first hair treatment condition is formulated as a shampoo, wherein the second hair treatment composition is formulated as a conditioner, and wherein the third hair treatment composition is formulated as a spray or foam. According to one embodiment, the shampoo includes a keratin protein composition as provided herein, water, red clover extract, sodium hyaluronate, red algae extract, rice oil glycereth-8 esters, and at least one biomimetic signal peptide such as acetyl tetrapeptide-3. According to one embodiment, the conditioner includes at least one biomimetic signal peptide such as acetyl tetrapeptide-3, Cyperus esculentus root oil, and at least one probiotic such as Saccharomyces lysate extract. According to one embodiment, the spray includes a keratin protein composition, water, red clover extract, at least one biomimetic signal peptide, at least one probiotic such as Saccharomyces lysate extract, larix/larch europaea wood extract, and methyl nicotinate. According to one embodiment, the hair treatment composition increases hair density by at least about 5%, 10%, 15%, 20%, 25%, 30%, 35%, 40%, 45%, or 50% after 120 days of daily administration of the first, second and third hair treatment composition. According to one embodiment, the hair treatment composition increases hair density by at least about 5%, 10%, 15%, 20%, 25%, 30%, 35%, 40%, 45%, or 50% after 180 days of daily administration of the first, second and third hair treatment composition. According to one embodiment, the hair treatment composition increases hair density by at least about 10% after 120 days of daily administration of the first, second and third hair treatment composition.

A method of increasing the percentage of anagen hair is provided that includes the step of administering an effective amount of at least one hair treatment composition as provided herein to a surface of the hair and scalp in need of treatment. According to one embodiment, the anagen hair count is increased by at least 5%, 10%, 15%, 20%, 25%, 30%, 35%, 40%, 45%, or 50% after 120 days of daily administration. According to one embodiment, the anagen hair count is increased by at least 5%, 10%, 15%, 20%, 25%, 30%, 35%, 40%, 45%, or 50%, 55%, 60%, 65%, 70%, 75%, or 80% after 180 days of daily administration. According to one embodiment, the anagen hair count is increased by at least 15% after 120 days of daily administration of the hair treatment composition. According to one embodiment, the anagen hair count is increased by at least 25% after 180 days of daily administration of the hair treatment composition.

A method of increasing the percentage of anagen hair is provided that includes the step of administering an effective amount of a hair treatment composition as provided herein to a surface of the hair and scalp in need of treatment, wherein the hair treatment composition is formulated as a shampoo. According to one embodiment, the anagen hair count is increased by at least 5%, 10%, 15%, 20%, 25%, 30%, 35%, 40%, 45%, or 50% after 120 days of daily administration. According to one embodiment, the anagen hair count is increased by at least 5%, 10%, 15%, 20%, 25%, 30%, 35%, 40%, 45%, or 50%, 55%, 60%, 65%, 70%, 75%, or 80% after 180 days of daily administration. According to one embodiment, the anagen hair count is increased by at least 15% after 120 days of daily administration of the hair treatment composition. According to one embodiment, the anagen hair count is increased by at least 25% after 180 days of daily administration of the hair treatment composition. According to one embodiment, the shampoo includes a keratin protein composition as provided herein, water, red clover extract, sodium hyaluronate, red algae extract, rice oil glycereth-8 esters, and at least one biomimetic signal peptide such as acetyl tetrapeptide-3.

A method of increasing the percentage of anagen hair is provided that includes the step of administering an effective amount of a hair treatment composition as provided herein to a surface of the hair and scalp in need of treatment, wherein the hair treatment composition is formulated as a conditioner. According to one embodiment, the anagen hair count is increased by at least 5%, 10%, 15%, 20%, 25%, 30%, 35%, 40%, 45%, or 50% after 120 days of daily administration. According to one embodiment, the anagen hair count is increased by at least 5%, 10%, 15%, 20%, 25%, 30%, 35%, 40%, 45%, or 50%, 55%, 60%, 65%, 70%, 75%, or 80% after 180 days of daily administration. According to one embodiment, the anagen hair count is increased by at least 15% after 120 days of daily administration of the hair treatment composition. According to one embodiment, the anagen hair count is increased by at least 25% after 180 days of daily administration of the hair treatment composition. According to one embodiment, the conditioner includes a keratin protein composition, water, red cover extract, at least one biomimetic signal peptide such as acetyl tetrapeptide-3, Cyperus Esculentus root oil, and at least one probiotic such as Saccharomyces lysate extract.

A method of increasing the percentage of anagen hair is provided that includes the step of administering an effective amount of a hair treatment composition as provided herein to a surface of the hair and scalp in need of treatment, wherein the hair treatment composition is formulated as a spray. According to one embodiment, the anagen hair count is increased by at least 5%, 10%, 15%, 20%, 25%, 30%, 35%, 40%, 45%, or 50% after 120 days of daily administration of the hair treatment composition. According to one embodiment, the anagen hair count is increased by at least 5%, 10%, 15%, 20%, 25%, 30%, 35%, 40%, 45%, or 50%, 55%, 60%, 65%, 70%, 75%, or 80% after 180 days of daily administration of the hair treatment composition. According to one embodiment, the anagen hair count is increased by at least 15% after 120 days of daily administration of the hair treatment composition. According to one embodiment, the anagen hair count is increased by at least 25% after 180 days of daily administration of the treatment composition. According to one embodiment, the spray includes a keratin protein composition, water, red clover extract, at least one biomimetic signal peptide, at least one probiotic such as saccharomyces lysate extract, larix/larch europaea wood extract, and methyl nicotinate.

A method of increasing the percentage of anagen hair is provided that includes the step of administering an effective amount of a hair treatment composition as provided herein to a surface of the hair and scalp in need of treatment, wherein the hair treatment composition is formulated as a foam. According to one embodiment, the anagen hair count is increased by at least 5%, 10%, 15%, 20%, 25%, 30%, 35%, 40%, 45%, or 50% after 120 days of daily administration of the hair treatment composition. According to one embodiment, the anagen hair count is increased by at least 5%, 10%, 15%, 20%, 25%, 30%, 35%, 40%, 45%, or 50%, 55%, 60%, 65%, 70%, 75%, or 80% after 180 days of daily administration of the hair treatment composition. According to one embodiment, the anagen hair count is increased by at least 15% after 120 days of daily administration of the hair treatment composition. According to one embodiment, the anagen hair count is increased by at least 25% after 180 days of daily administration of the treatment composition. According to one embodiment, the foam includes a keratin protein composition as provided herein, water, panthenol, plant extracts, and microalgae extract.

A method of increasing or otherwise promoting an increase in anagen hair or increase in the percentage of anagen hair is provided that includes the steps of: administering an effective amount of a first hair treatment composition as provided herein to a surface of the hair and scalp in need of treatment; administering an effective amount of a second hair treatment composition as provided herein to a surface of the hair and scalp in need of treatment; and administering an effective amount of a third hair treatment composition as provided herein to a surface of the hair and scalp in need of treatment, wherein the first hair treatment condition is formulated as a shampoo, wherein the second hair treatment composition is formulated as a conditioner, and wherein the third hair treatment composition is formulated as a spray or foam. According to one embodiment, the shampoo includes a keratin protein composition as provided herein, water, red clover extract, sodium hyaluronate, red algae extract, rice oil glycereth-8 esters, and at least one biomimetic signal peptide such as acetyl tetrapeptide-3. According to one embodiment, the conditioner includes at keratin protein composition, water, red cover extract, at least one biomimetic signal peptide such as acetyl tetrapeptide-3, Cyperus esculentus root oil, and at least one probiotic such as saccharomyces lysate extract. According to one embodiment, the spray includes a keratin protein composition, water, red clover extract, at least one biomimetic signal peptide, at least one probiotic such as saccharomyces lysate extract, larix/larch europaea wood extract, and methyl nicotinate. According to one embodiment, the anagen hair count is increased by at least 5%, 10%, 15%, 20%, 25%, 30%, 35%, 40%, 45%, or 50% after 120 days of daily administration of the first, second and third hair treatment composition. According to one embodiment, the anagen hair count is increased by at least 5%, 10%, 15%, 20%, 25%, 30%, 35%, 40%, 45%, or 50%, 55%, 60%, 65%, 70%, 75%, or 80% after 180 days of daily administration of the first, second and third hair treatment composition. According to one embodiment, the anagen hair count is increased by at least 15% after 120 days of daily administration of the first, second and third hair treatment composition. According to one embodiment, the anagen hair count is increased by at least 25% after 180 days of daily administration of the first, second and third hair treatment composition. According to one embodiment, the increase in the percentage of anagen hair is due to new anagen hairs. According to one embodiment, the increase in the percentage of anagen hair is due to the maintenance of or increase in existing anagen hairs.

A method of supporting or otherwise promoting hair growth is provided that includes the steps of: administering an effective amount of a first hair treatment composition as provided herein to a surface of the hair and scalp in need of treatment; administering an effective amount of a second hair treatment composition as provided herein to a surface of the hair and scalp in need of treatment; and administering an effective amount of a third hair treatment composition as provided herein to a surface of the hair and scalp in need of treatment, wherein the first hair treatment condition is formulated as a shampoo, wherein the second hair treatment composition is formulated as a conditioner, and wherein the third hair treatment composition is formulated as a spray or foam. According to one embodiment, the shampoo includes a keratin protein composition as provided herein, water, red clover extract, sodium hyaluronate, red algae extract, rice oil glycereth-8 esters, and at least one biomimetic signal peptide such as acetyl tetrapeptide-3, According to one embodiment, the conditioner includes a keratin protein composition, water, red cover extract, at least one biomimetic signal peptide such as acetyl tetrapeptide-3, Cyperus esculentus root oil, and at least one probiotic such as saccharomyces lysate extract. According to one embodiment, the spray includes a keratin protein composition, water, red clover extract, at least one biomimetic signal peptide, at least one probiotic such as saccharomyces lysate extract, larix/larch europaea wood extract, and methyl nicotinate. According to one embodiment, the anagen hair count is increased by at least 5%, 10%, 15%, 20%, 25%, 30%, 35%, 40%, 45%, or 50% after 120 days of daily administration of the first, second and third hair treatment composition. According to one embodiment, the anagen hair count is increased by at least 5%, 10%, 15%, 20%, 25%, 30%, 35%, 40%, 45%, or 50%, 55%, 60%, 65%, 70%, 75%, or 80% after 180 days of daily administration of the first, second and third hair treatment composition. According to one embodiment, the hair growth is new hair. According to one embodiment, the hair growth is existing hair.

A method of increasing the ratio of anagen phase hair to telogen phase hair in a subject in need of treatment is provided that includes the step of administering an effective amount of one or more hair treatment compositions as provided herein to a surface of the hair and scalp in need of treatment. According to one embodiment, the hair treatment composition increases the ratio of anagen phase hair to telogen phase hair by at least about 5%, 10%, 15%, 20%, 25%, 30%, 35%, 40%, 45%, or 50% after 120 days of daily administration. According to one embodiment, the hair treatment composition increases the ratio of anagen phase hair to telogen phase hair by at least about 5%, 10%, 15%, 20%, 25%, 30%, 35%, 40%, 45%, 50% or 55% after 180 days of daily administration. According to one embodiment, the hair treatment composition increases the ratio of anagen phase hair to telogen phase hair by at least about 10% after 120 days of daily administration of the hair treatment composition. According to one embodiment, the hair treatment composition increases the ratio of anagen phase hair to telogen phase hair by at least about 50% after 180 days of daily administration of the hair treatment composition.

A method of increasing the ratio of anagen phase hair to telogen phase hair in a subject in need of treatment is provided that includes the step of administering an effective amount of one or more hair treatment composition as provided herein to a surface of the hair and scalp in need of treatment, wherein the hair treatment composition is formulated as a shampoo. According to one embodiment, the hair treatment composition increases the ratio of anagen phase hair to telogen phase hair by at least about 5%, 10%, 15%, 20%, 25%, 30%, 35%, 40%, 45%, or 50% after 120 days of daily administration. According to one embodiment, the hair treatment composition increases the ratio of anagen phase hair to telogen phase hair by at least about 5%, 10%, 15%, 20%, 25%, 30%, 35%, 40%, 45%, 50% or 55% after 180 days of daily administration. According to one embodiment, the hair treatment composition increases the ratio of anagen phase hair to telogen phase hair by at least about 10% after 120 days of daily administration of the first, second and third hair treatment composition. According to one embodiment, the hair treatment composition increases the ratio of anagen phase hair to telogen phase hair by at least about 50% after 180 days of daily administration of the first, second and third hair treatment composition.

A method of increasing the ratio of anagen phase hair to telogen phase hair in a subject in need of treatment is provided that includes the step of administering an effective amount of one or more hair treatment composition as provided herein to a surface of the hair and scalp in need of treatment, wherein the hair treatment composition is formulated as a conditioner. According to one embodiment, the hair treatment composition increases the ratio of anagen phase hair to telogen phase hair by at least about 5%, 10%, 15%, 20%, 25%, 30%, 35%, 40%, 45%, or 50% after 120 days of daily administration. According to one embodiment, the hair treatment composition increases the ratio of anagen phase hair to telogen phase hair by at least about 5%, 10%, 15%, 20%, 25%, 30%, 35%, 40%, 45%, 50% or 55% after 180 days of daily administration. According to one embodiment, the hair treatment composition increases the ratio of anagen phase hair to telogen phase hair by at least about 10% after 120 days of daily administration of the first, second and third hair treatment composition. According to one embodiment, the hair treatment composition increases the ratio of anagen phase hair to telogen phase hair by at least about 50% after 180 days of daily administration of the first, second and third hair treatment composition.

A method of increasing the ratio of anagen phase hair to telogen phase hair in a subject in need of treatment is provided that includes the step of administering an effective amount of one or more hair treatment composition as provided herein to a surface of the hair and scalp in need of treatment, wherein the hair treatment composition is formulated as a spray. According to one embodiment, the hair treatment composition increases the ratio of anagen phase hair to telogen phase hair by at least about 5%, 10%, 15%, 20%, 25%, 30%, 35%, 40%, 45%, or 50% after 120 days of daily administration. According to one embodiment, the hair treatment composition increases the ratio of anagen phase hair to telogen phase hair by at least about 5%, 10%, 15%, 20%, 25%, 30%, 35%, 40%, 45%, 50% or 55% after 180 days of daily administration. According to one embodiment, the hair treatment composition increases the ratio of anagen phase hair to telogen phase hair by at least about 10% after 120 days of daily administration of the first, second and third hair treatment composition. According to one embodiment, the hair treatment composition increases the ratio of anagen phase hair to telogen phase hair by at least about 50% after 180 days of daily administration of the first, second and third hair treatment composition.

A method of increasing the ratio of anagen phase hair to telogen phase hair in a subject in need of treatment is provided that includes the step of administering an effective amount of one or more hair treatment composition as provided herein to a surface of the hair and scalp in need of treatment, wherein the hair treatment composition is formulated as a foam. According to one embodiment, the hair treatment composition increases the ratio of anagen phase hair to telogen phase hair by at least about 5%, 10%, 15%, 20%, 25%, 30%, 35%, 40%, 45%, or 50% after 120 days of daily administration. According to one embodiment, the hair treatment composition increases the ratio of anagen phase hair to telogen phase hair by at least about 5%, 10%, 15%, 20%, 25%, 30%, 35%, 40%, 45%, 50% or 55% after 180 days of daily administration. According to one embodiment, the hair treatment composition increases the ratio of anagen phase hair to telogen phase hair by at least about 10% after 120 days of daily administration of the first, second and third hair treatment composition. According to one embodiment, the hair treatment composition increases the ratio of anagen phase hair to telogen phase hair by at least about 50% after 180 days of daily administration of the first, second and third hair treatment composition.

A method of increasing the ratio of anagen phase hair to telogen phase hair in a subject in need of treatment is provided that includes the steps of: administering an effective amount of a first hair treatment composition as provided herein to a surface of the hair and scalp in need of treatment; administering an effective amount of a second hair treatment composition as provided herein to a surface of the hair and scalp in need of treatment; and administering an effective amount of a third hair treatment composition as provided herein to a surface of the hair and scalp in need of treatment, wherein the first hair treatment condition is formulated as a shampoo, wherein the second hair treatment composition is formulated as a conditioner, and wherein the third hair treatment composition is formulated as a spray or foam. According to one embodiment, the shampoo includes a keratin protein composition as provided herein, water, red clover extract, sodium hyaluronate, red algae extract, rice oil glycereth-8 esters, and at least one biomimetic signal peptide such as acetyl tetrapeptide-3. According to one embodiment, the conditioner includes a keratin protein composition, water, red cover extract, at least one biomimetic signal peptide such as acetyl tetrapeptide-3, Cyperus esculentus root oil, and at least one probiotic such as saccharomyces lysate extract. According to one embodiment, the spray includes a keratin protein composition, water, red clover extract, at least one biomimetic signal peptide, at least one probiotic such as saccharomyces lysate extract, larix/larch europaea wood extract, and methyl nicotinate. According to one embodiment, the hair treatment composition increases the ratio of anagen phase hair to telogen phase hair by at least about 5%, 10%, 15%, 20%, 25%, 30%, 35%, 40%, 45%, or 50% after 120 days of daily administration of the first, second and third hair treatment composition. According to one embodiment, the hair treatment composition increases the ratio of anagen phase hair to telogen phase hair by at least about 5%, 10%, 15%, 20%, 25%, 30%, 35%, 40%, 45%, 50% or 55% after 180 days of daily administration of the first, second and third hair treatment composition. According to one embodiment, the hair treatment composition increases the ratio of anagen phase hair to telogen phase hair by at least about 10% after 120 days of daily administration of the first, second and third hair treatment composition. According to one embodiment, the hair treatment composition increases the ratio of anagen phase hair to telogen phase hair by at least about 50% after 180 days of daily administration of the first, second and third hair treatment composition.

A method of decreasing hair thinning is provided that includes the steps of: administering an effective amount of a first hair treatment composition as provided herein to a surface of the hair and scalp in need of treatment; administering an effective amount of a second hair treatment composition as provided herein to a surface of the hair and scalp in need of treatment; and administering an effective amount of a third hair treatment composition as provided herein to a surface of the hair and scalp in need of treatment, wherein the first hair treatment condition is formulated as a shampoo, wherein the second hair treatment composition is formulated as a conditioner, and wherein the third hair treatment composition is formulated as a spray or foam. According to one embodiment, the shampoo includes a keratin protein composition as provided herein, water, red clover extract, sodium hyaluronate, red algae extract, rice oil glycereth-8 esters, and at least one biomimetic signal peptide such as acetyl tetrapeptide-3. According to one embodiment; the conditioner includes a keratin protein composition, water, red cover extract, at least one biomimetic signal peptide such as acetyl tetrapeptide-3, Cyperus esculentus root oil, and at least one probiotic such as saccharomyces lysate extract. According to one embodiment, the spray includes a keratin protein composition, water, red clover extract, at least one biomimetic signal peptide, at least one probiotic such as saccharomyces lysate extract, larix/larch europaea wood extract, and methyl nicotinate. According to one embodiment, the hair thinning is caused by aging.

A method of decreasing hair shedding is provided that includes the steps of: administering an effective amount of a first hair treatment composition as provided herein to a surface of the hair and scalp in need of treatment; administering an effective amount of a second hair treatment composition as provided herein to a surface of the hair and scalp in need of treatment; and administering an effective amount of a third hair treatment composition as provided herein to a surface of the hair and scalp in need of treatment, wherein the first hair treatment condition is formulated as a shampoo, wherein the second hair treatment composition is formulated as a conditioner, and wherein the third hair treatment composition is formulated as a spray or foam. According to one embodiment, the shampoo includes a keratin protein composition as provided herein, water, red clover extract, sodium hyaluronate, red algae extract, rice oil glycereth-8 esters, and at least one biomimetic signal peptide such as acetyl tetrapeptde-3. According to one embodiment, the conditioner includes a keratin protein composition, water, red cover extract, at least one biomimetic signal peptide such as acetyl tetrapeptide-3, Cyperus esculentus root oil, and at least one probiotic such as saccharomyces lysate extract. According to one embodiment, the spray includes a keratin protein composition, water, red clover extract, at least one biomimetic signal peptide, at least one probiotic such as saccharomyces lysate extract, larix/larch europaea wood extract, and methyl nicotinate.

According to another aspect, a method for maintaining and improving scalp microbiome is provided. The method includes the steps of administering an effective amount of a hair treatment composition as provided herein to a surface of the scalp in need of treatment thereby improving the scalp microbiome. By improving scalp microbiome, common scalp conditions such as flaking, seborrheic dermatitis, dandruff, and psoriasis are treated or otherwise prevented. The hair treatment composition as provided herein aids in the maintenance and improvement of the scalp microbiome by aiding in the balance of the various microorganisms that reside on the surface scalp including fungi, yeast and bacteria.

A method of promoting scalp microbiome health in a subject is provided. The method includes the step of administering, applying or otherwise introducing an effective amount of at least one hair treatment composition to a surface of the scalp in need of treatment. According to one embodiment, the at least one hair treatment composition is a shampoo, conditioner, spray, foam or any combination thereof, as described herein. By promoting scalp microbiome health, the hair treatment composition as provided herein may increase or encourage the growth of certain beneficial microorganisms.

A method of improving scalp flora ecology in a subject is provided. The method includes the step of administering, applying or otherwise introducing an effective amount of at least one hair treatment composition to a surface of the scalp in need of treatment. According to one embodiment, the at least one hair treatment composition is a shampoo, conditioner, spray, foam or any combination thereof, as described herein.

A method of maintaining scalp microbiome diversity in a subject is provided. The method includes the step of administering, applying or otherwise introducing an effective amount of at least one hair treatment composition to a surface of the scalp in need of treatment. According to one embodiment, the at least one hair treatment composition is a shampoo, conditioner, spray, foam or any combination thereof, as described herein. The diversity of microbiome species denotes balance and health because of the increase in beneficial metabolites. The commensal skin microbiota plays an important role in influencing the immune response from the skin and acts as a barrier against the colonization of certain microorganisms including potentially pathogens and the overgrowth of opportunistic pathogens.

A method of promoting scalp microbiome health in a subject by increasing scalp pH is provided. The method includes the step of administering an effective amount of a hair treatment composition to a surface of the scalp in need of treatment. According to one embodiment, the pH of the scalp is increased by at least about 10% after 28 days of daily administration. According to one embodiment, the pH of the scalp is increased above the pH of hair or above about 3.6. According to one embodiment, the pH of the scalp is maintained between 4.5 and 5.5. According to one embodiment, the pH of the scalp is maintained between 4.5 and 5.5 after 28 days of daily administration.

A hair treatment regimen is provided that includes the steps of: administering an effective amount of a first hair treatment composition as provided herein to a surface of the hair and scalp in need of treatment; administering an effective amount of a second hair treatment composition as provided herein to a surface of the hair and scalp in need of treatment; and administering an effective amount of a third hair treatment composition as provided herein to a surface of the hair and scalp in need of treatment, wherein the first hair treatment condition is formulated as a shampoo, wherein the second hair treatment composition is formulated as a conditioner, and wherein the third hair treatment composition is formulated as a spray or foam. According to one embodiment, the shampoo includes a keratin protein composition as provided herein, water, red clover extract, sodium hyaluronate, red algae extract, rice oil glycereth-8 esters, and at least one biomimetic signal peptide such as acetyl tetrapeptide-3. According to one embodiment, the conditioner includes a keratin protein composition, water, red cover extract, at least one biomimetic signal peptide such as acetyl tetrapeptide-3, Cyperus esculentus root oil, and at least one probiotic such as saccharomyces lysate extract. According to one embodiment, the spray includes a keratin protein composition, water, red clover extract, at least one biomimetic signal peptide, at least one probiotic such as saccharomyces lysate extract, larix/larch europaea wood extract, and methyl nicotinate. According to one embodiment, wherein the regimen increases anagen hair count by at least 5%, 10%, 15%, 20%, 25%, 30%, 35%, 40%, 45%, or 50% after 120 days of daily administration of the first, second and third hair treatment composition. According to one embodiment, wherein the regimen increases anagen hair count by at least 5%, 10%, 15%, 20%, 25%, 30%, 35%, 40%, 45%, or 50%, 55%, 60%, 65%, 70%, 75%, or 80% after 180 days of daily administration of the first, second and third hair treatment composition.

According to another aspect, a method for reducing hair breakage is provided. The method includes the steps of administering an effective amount of a hair treatment composition as provided herein to a surface of the hair in need of treatment thereby increasing hair thickness and hair volume to reduce hair breakage. The hair may be styled immediately after application. The hair may also be washed between applications. According to one embodiment, the hair breakage is reduced by at least 40% after one administration.

According to another aspect, a method for increasing hair strength is provided. The method includes the steps of administering an effective amount of a hair treatment composition as provided herein to a surface of the hair in need of treatment thereby increasing hair strength. The hair may be styled immediately after application. The hair may also be washed between applications.

According to another aspect, a method for repairing hair damage is provided. The method includes the steps of administering an effective amount of a hair treatment composition as provided herein to a surface of the hair in need of treatment thereby repairing any damage that may be in or on the hair. The hair may be styled immediately after application. The hair may also be washed between applications.

According to another aspect, a method of reducing hair frizziness is provided. The method includes the step of administering an effective amount of a hair treatment composition as provided herein to a surface of the hair in need of treatment thereby causing the hair to exhibit less frizz. The hair may be styled immediately after application. The hair may also be washed between applications. According to one embodiment, the hair frizziness is reduced by at least 40%.

According to another aspect, a method of reducing or mending hair split ends is provided. The method includes the steps of administering an effective amount of a hair treatment composition as provided herein to a surface of the hair in need of treatment thereby resulting in split end mending or reducing the number of split ends. The hair may be styled immediately after application. The hair may also be washed between applications. According to one embodiment, at least 80% of the treated split end hair is completely mended according to the formula:

$$\% mended = \frac{[ \# of mended fibers + \# of partially mended fibers ]}{Total \# of split end fibers taken initially} \times 100.$$

According to another aspect, a method of increasing hair strand thickness is provided. The method includes the step of administering, applying or otherwise introducing an effective amount of a hair treatment composition to the surface of a mammal's hair in need of treatment.

According to another aspect, a method of increasing hair strand volume is provided. The method includes the step of administering, applying or otherwise introducing an effective amount of a hair treatment composition to the surface of a mammal's hair in need of treatment.

According to another aspect, a method of retaining hair gloss is provided. The method includes the step of administering, applying or otherwise introducing an effective amount of a hair treatment composition to the surface of a mammal's hair in need of treatment.

According to another aspect, a method of improving hair color concentration is provided. The method includes the step of administering, applying or otherwise introducing an effective amount of a hair treatment composition to the surface of a mammal's hair in need of treatment.

According to one embodiment, a method of reducing scalp irritation, scalp inflammation or scalp pain is provided. The method includes the step of administering, applying or otherwise introducing an effective amount of a hair treatment composition to the surface of a mammal's scalp that is in need of treatment.

The disclosure having been generally described, the following examples are given as particular embodiments of the disclosure and to demonstrate the practice and advantages thereof. It is understood that the examples are given by way of illustration and are not intended to limit the specification or the claims in any manner.

### Example 1

### Randomized Clinical Evaluation of Hair Treatment Regimen and Minoxidil Regimen

A randomized clinical evaluation was conducted by an independent third party laboratory to assess a Hair Treatment Regimen and Minoxidil Regimen efficacy in improving hair volume, increasing the percentage of hair in the anagen phase and in improving the hair loss reduction, under normal use condition. The Hair Treatment Regimen and Minoxidil Regimen utilized in the clinical evaluation are provided and described below.

### Hair Treatment Regimen

The Hair Treatment Regimen included daily administration of each of the Shampoo (Table 1), Conditioner (Table 2) and Density Treatment Composition (Table 3).

During the clinical evaluation, the Shampoo was applied to wet hair, massaged into a rich lather, and rinsed. The Conditioner was then applied to the wet hair evenly and rinsed. Finally, the Density Treatment Composition, formulated as a spray, was applied or otherwise sprayed directly onto scalp in the hair loss areas when the hair was dry or damp and lifted to allow application. The Density Treatment Composition was then massaged in and not rinsed. This Hair Treatment Regimen was repeated daily in the evening.

TABLE 1
Shampoo

| | Amount |
|---|---|
| Component | (% w/w) |
| Water | To QS |
| | 100% |
| Keratin Protein Composition | 1% |
| (about 95% w/w alpha keratin and about 5% w/w gamma | to 7% |
| keratin) | |
| Trifolium Pratense (Red Clover) Flower Extract | 1 |
| | to 10% |
| Sodium Hyaluronate | 0.1% |
| | to 5% |
| Red Algae Extract | 3% |
| | to 7% |
| Rice-based humectants-Rice Oil Glycereth-8 Esters | 0.1% |
| | to 5% |
| Biomimetic Signal Peptide-Acetyl Tetrapeptide-3 | 0.1% |
| | to 7% |
| Sodium Lauroyl Methyl Isethionate, Sodium Cocoyl | Less |
| Isethionate, Lauryl Glucoside, Acrylates Copolymer, | than |
| Camellia Sinensis Leaf Extract, Larix Europaea Wood | 70.0% |
| Extract, Tocopherol, Sodium Phytate, Cocamide MIPA, | |
| Polysorbate 20, Hydrogenated Coconut Acid, Sodium | |
| Isethionate, Sodium Methyl Cocoyl Taurate, Caprylyl | |
| Glycol, Guar Hydroxypropyltrimonium Chloride, Trisodium | |
| Ethylenediamine Disuccinate, Ethylhexylglycerin, Hexylene | |
| Glycol, Glycerin, Butylene Glycol, Sodium | |
| Dilauramidoglutamide Lysine, Isopropanolamine, Coconut | |
| Acid, Dextran, Chondrus Crispus Extract, Ethylhexyl | |
| Salicylate, Sodium Metabisulfite, Glycine, Gluconolactone, | |
| Zinc Chloride, Phenoxyethanol, Parfum (Fragrance), | |
| Potassium Sorbate, Sodium Hydroxide, Citric Acid, Sodium | |
| Benzoate, Sodium Chloride, Linalool, Geraniol, Limonene, | |
| Citronellol | |

TABLE 2
Conditioner

| Component | Amount |
|---|---|
| Water | To QS |
| | 100% |
| Keratin Protein Composition | 1% |
| (about 95% w/w alpha keratin and about 5% w/w gamma | to 7% |
| keratin) | |
| Trifolium Pratense (Red Clover) Flower Extract | 1 |
| | to 10% |
| Biomimetic Signal Peptide-Acetyl Tetrapeptide-3 | 0.1% |
| | to 7% |
| Cyperus Esculentus Root Oil | 0.1% |
| | to 5% |
| Probiotic-Saccharomyces Lysate Extract, | 1% |
| | to 7% |
| Cetearyl Alcohol, Dimethicone, Brassicamidopropyl | Less |
| Dimethylamine, Cetyl Esters, Cetyl Alcohol, | than |
| Behentrimonium Chloride, Isododecane, Ethylhexylglycerin, | 70% |
| PEG-100 Stearate, Glyceryl Stearate, Silybum Marianum | |
| Ethyl Ester, Panthenol, Tocopheryl Nicotinate, Tocopherol, | |
| Madecassoside, Quercus Petraea Fruit Extract, Citrus | |
| Paradisi (Grapefruit) Fruit Extract, Asiaticoside, Hydrolyzed | |
| Adansonia Digitata Seed Extract, Amaranthus | |
| Hypochondriacus Leaf Extract, Larix Europaea Wood | |
| Extract, Glycine, Hydrolyzed Vegetable Protein, Hydrolyzed | |
| Quinoa, Oryza Sativa (Rice) Extract, Hydrolyzed Linseed | |
| Extract, Camellia Sinensis Leaf Extract, Rosmarinus | |
| Officinalis (Rosemary) Leaf Extract, Hydrogenated | |
| Ethylhexyl Olivate, Oryza Sativa (Rice) Bran Extract, | |
| Hydrogenated Olive Oil Unsaponifiables, Jojoba Esters, | |
| Helianthus Annuus (Sunflower) Seed Oil, Oryza Sativa | |
| (Rice) Seed Protein, Phytic Acid, Tetrasodium Glutamate | |
| Diacetate, Butylene Glycol, Sodium Dilauramidoglutamide | |
| Lysine, Dextran, Disodium Phosphate, Caprylyl Glycol, | |
| Propanediol, Parfum (Fragrance), Hydroxyethylcellulose, | |
| Polysorbate 60, Benzyl Alcohol, Polysorbate 20, Sorbic | |
| Acid, Benzoic Acid, Glycerin, Phenoxyethanol, | |
| Dimethiconol, Chlorphenesin, Sodium Metabisulfite, | |
| Gluconolactone, Zinc Chloride, Sodium Hydroxide, Alcohol, | |
| Citric Acid, Tetrasodium EDTA, Sodium Benzoate, Sodium | |
| Phosphate, Potassium Sorbate, Calcium Gluconate, BHT, | |
| Linalool, Citronellol, Geraniol, Limonene. | |

TABLE 3
Hair Density Treatment Composition (Spray)

| Component | Amount |
|---|---|
| Water | To QS |
| | 100% |
| Keratin Protein Composition | 1% |
| (about 95% w/w alpha keratin and about 5% w/w gamma | to 7% |
| keratin) | |
| Biomimetic Signal Peptide-Acetyl Tetrapeptide-3 | 0.1% |
| | to 7% |
| Trifolium Pratense (Red Clover) Flower Extract | 1 |
| | to 10% |
| Saccharomyces Lysate Extract | 1% |
| | to 7% |
| Larix/Larch Europaea Wood Extract | 1% |
| | to 7% |
| Methyl Nicotinate | 0.5% |
| | to 3% |
| Zingiber Officinale (Ginger) Root Extract, Pisum Sativum | Less |
| (Pea) Extract, Nasturtium Officinale Extract, Moringa | than |
| Oleifera Seed Extract, Jasminum Officinale (Jasmine) | 80% |
| Flower Extract, Hydrolyzed Linseed Extract, Hydrolyzed | |
| Hibiscus Esculentus Extract, Helianthus Annuus | |
| (Sunflower) Seed Extract, Daucus Carota Sativa (Carrot) | |
| Root Extract, Cynara Scolymus (Artichoke) Leaf Extract, | |
| Amaranthus Caudatus Seed Extract, Camellia Sinensis | |
| Leaf Extract, Isochrysis Galbana Extract, Tocopherol, | |
| Mentha Piperita (Peppermint) Oil, Dextran, Pentylene | |
| Glycol, Sodium Phytate, Sodium Dilauramidoglutamide | |
| Lysine, Quaternium-51, Ethylhexylglycerin, Sodium | |
| Metabisulfite, Glycine, Parfum (Fragrance), Biosaccharide | |
| Gum-4, Butylene Glycol, Glycerin, Zinc Chloride, PPG-26- | |
| Buteth-26, Propanediol, Polyacrylate Crosspolymer-6, | |
| PEG-40 Hydrogenated Castor Oil, t-Butyl Alcohol, Alcohol, | |
| Tetrasodium EDTA, Sodium Benzoate, Potassium Sorbate, | |
| Disodium EDTA, Phenoxyethanol, Citric Acid, Linalool, | |
| Geraniol, Limonene, Citronellol, Benzyl Benzoate, Citral, | |
| Hexyl Cinnamal | |

### Minoxidil Regimen

The Shampoo (Table 1) was applied to wet hair, massaged into a rich lather, and rinsed. The Conditioner (Table 2) was then applied to the wet hair evenly and rinsed. A minoxidil topical aerosol (5%) was directly applied or otherwise sprayed on dry scalp in the hair loss area(s) and massaged into the scalp with fingers. This Minoxidil Regimen was repeatedly daily in the evening.

### Clinical Evaluation Methods

Hair loss products can be evaluated through efficacy studies that allow independent laboratories to assess a hair loss product's characteristics, detecting complaints and comments regarding performance, as well as testing the quality control and the assured and claims support (what the product offers). The claims must be supported by solid, clear and relevant evidences. Such evidences may result from consumers evaluations (ASTM E 1958-06, 2006).

For the efficacy assessment of products, clinical and/or self-assessment studies can be used. Self-assessment by test subjects is performed by following the “Standard Guide for Sensory Claim Substantiation” (ASTM E 1958-06, 2006), by using questionnaires. The ASTM (American Society for Testing and Materials) standards organization has been developed for over a century and represents one of the greatest voluntary organizations for standards development in the world, being a reliable source of technical standards of material, products, systems and services. The “Standard Guide for Sensory Claim Substantiation” is an ASTM standard that aims to disclose the good practices in sensory studies, approaching reasonable practices for executing sensory studies to validate product claims. Subjective questionnaires gauge the test subjects' perceptions of the investigational products and its effects. Questions were asked for test subjects' agreement to a statement with a seven-point scale as follows: 7—strongly agree; 6—agree; 5—somewhat agree; 4—neither agree nor disagree; 4—somewhat disagree; 3—somewhat disagree; 2—disagree; and 1—strongly disagree.

### Randomized Clinical Evaluation

The randomized clinical evaluation was conducted by an independent third party laboratory to assess the Hair Treatment Regimen and Minoxidil Regimen efficacy in improving hair volume, improving hair growth and in improving hair loss reduction (reducing hair loss), under normal use condition, through the following the methodologies:
Phototrichogram analysis;
Digital Photographic Documentation;
Subjective self-perception of the study subject by Self-Assessments.

A total of 65 test subjects were included in the clinical evaluation. The test subjects were women between 35 and 59 years old that were perceived to have suffered from excessive hair shedding, thinning hair and/or receding hairline. A total of 35 test subjects were treated with the Hair Treatment Regimen provided herein. A total of 30 test subjects were treated with the Minoxidil Regimen provided herein.

At the initial visit (referred to as “T0-D0”), the test subjects were informed about the study objective, methodology, duration, and the possibly expected benefits and constraints related to the study. The appropriate consents were obtained. A dermatologist carried out a clinical assessment, verification of compliance with the requirements of the study, and in the subsequent visits carried out a verification of possible adverse events, discomfort sensations and confirmation of the correct use of the products.

Phototrichogram analysis performed is a non-invasive technique to perform quantitative measurements of hair health such as via hair density and the percent of follicles in the anagen phase (anagen %). An area of with 1.0 cm² on the top of the test subject's head (vertex) was marked for the phototrichogram analysis. The delimited area was an area affected by the hair loss. The shaving was made with the aid of a clipper and then liquid Vaseline® was applied on the area for a better contrast of the photograph. Using a camera Nikon® with an Epiflash Canfield system for phototrichogram, the images were captured. The phototrichogram images were used by a trained technician for hair count through the software Adobe® Photoshop CS4. The following parameters were evaluated: number of anagen hair; anagen/telogen ratio; and total density. The phototrichogram analysis were performed at the following time-points: T0-D0 before regimens; T0-D2 48 hours after the shaving; T120-D0 after 4 months of investigational products use; T120-D2 after 4 months of investigational products use and 48 hours after the shaving; T180-D0 after 6 months of investigational products use; and T180-D2 after 6 months of investigational products use and 48 hours after the shaving.

Digital images were taken of all test subjects' head (vertex) in a standardized way. Each test subject's hair was parted down the center. The test subject's head was, then, positioned in a Canfield Scientific hair photography device for full head/hair image capture. Test subjects' hair were off their face, jewelry removed, and a black drape used to standardize clothing. Digital images were taken using a digital camera Canon® 60D A-EG-151 and fixed lighting of the mid-pattern and vertex views. Images of full head at area of thinning were taken with an overhead shot, as well as left and right-side views, of each female test subject at each check-in. Digital images of the left and right-side hair views were captured using the IntelliStudio® System (Canfield Scientific, Inc.). The images were capture for documentation at the following time-points: T0-D0 before regimens; T120-D0 after 4 months of investigational products use; and T180-D0 after 6 months of investigational products use.

After initial evaluations, subjects were asked to fill out a self-assessment questionnaire (T0-D0) and received the respective Regimen for use at home, under normal conditions of use, for 6 months and the products daily log. The results of the initial self-assessment questionnaire are provided in Table 4. After the initial self-assessment, test subjects were instructed to record in the daily log of the products used for all the applications performed and possible comments about the products. Subjects returned to the Institute 48 hours after the shaving (T0-D2) and new images were collected for phototrichogram analysis.

After 45 days of use (referred to as “T45”) and 90 days of use (referred to as “T90”) of the Hair Treatment Regimen and Minoxidil regimen, test subjects were called to check compliance with the study and also to check for possible signs of discomfort. At time-point T90, the subjects also answered to a self-assessment questionnaire by phone.

TABLE 4
T0-D0

| | Positive | Positive |
|---|---|---|
| | Response | Response |
| | with Hair | with |
| | Treatment | Minoxidil |
| Statement | Regimen (%) | Regimen (%) |
| My hair has body | 68.6% | 53.3% |
| My hair has volume | 68.8% | 40.0% |
| My hair looks full | 60.0% | 33.3% |
| My hair feels strong | 60.0% | 30.0% |
| My hair feels thick | 37.1% | 10.0% |
| My hair is easy to style | 91.4% | 73.3% |
| My hair feels healthy | 80.0% | 46.7% |
| My scalp feels healthy | 85.7% | 63.3% |
| My hair has damage | 45.7% | 73.3% |
| I'm noticing more shedding/ | 40.0% | 83.3% |
| fallout than I used to | | |
| My scalp is more visible | 88.6% | 86.7% |
| than it used to be | | |
| My scalp has areas of irritation | 5.7% | 0% |
| I feel good about my hair | 71.4% | 33.3% |
| I feel confident in my appearance | 71.4% | 50.0% |
| I feel beautiful | 80.0% | 63.3% |

After 4 months (T120-D0) and 6 months (T180-D0) of use, the subjects returned and a new shaving and phototrichrogram analyses were performed on the previously determined area. The test subjects were asked to answer the self-assessment questionnaire and digital images were taken for photographic documentation using a digital camera Canon® 60D A-EG-151. Test subjects returned 48 hours after the shaving (T120-D2 and T180-D2) and a new phototrichogram analysis were performed. During all visits, test subjects remained at rest in a room with controlled temperature and humidity for, at least, 30 minutes before and during all procedures. The questionnaire results at 120 days (T120-D0) is show in Table 5. The questionnaire results at 180 days (T180-D0) is show in Table 6.

TABLE 5
T120-D0

| | Positive | Positive |
|---|---|---|
| | Response | Response |
| | with Hair | with |
| | Treatment | Minoxidil |
| Statement | Regimen (%) | Regimen (%) |
| My hair has more volume | 80.0% | 96.7% |
| My hair has more body | 77.1% | 96.7% |
| My hair looks fuller | 85.7% | 96.7% |
| My hair feels stronger | 82.9% | 96.7% |
| My hair feels thicker | 71.4% | 73.3% |
| My hair feel healthier | 85.7% | 96.7% |
| My hair is easier to style | 85.7% | 86.7% |
| My hair is less damaged | 85.7% | 96.7% |
| I notice my existing hair | 85.7% | 96.7% |
| growing faster | | |
| I notice less shedding/fall out | 91.4% | 96.7% |
| My scalp is less irritated | 77.1% | 93.3% |
| My scalp feels healthier | 85.7% | 100.0% |
| My scalp is less visible | 82.9% | 96.7% |
| I see new hair growth | 94.3% | 100.0% |
| I feel better about my hair | 94.3% | 100.0% |
| I feel more confident in my | 94.3% | 93.3% |
| appearance | | |
| I feel more beautiful | 91.4% | 96.7% |
| This regimen is more effective | 85.7% | 100.0% |
| than hair growth solutions I've | | |
| tried in the past | | |

TABLE 6
T180-D0

| | Positive | Positive |
|---|---|---|
| | Response | Response |
| | with Hair | with |
| | Treatment | Minoxidil |
| Statement | Regimen (%) | Regimen (%) |
| My hair has more volume | 94.3% | 96.7% |
| My hair has more body | 91.4% | 96.7% |
| My hair looks fuller | 94.3% | 96.7% |
| My hair feels stronger | 91.4% | 96.7% |
| My hair feels thicker | 82.9% | 83.3% |
| My hair feels healthier | 94.3% | 100.0% |
| My hair is easier to style | 88.6% | 96.7% |
| My hair is less damaged | 85.7% | 96.7% |
| I notice my existing hair | 100.0% | 100.0% |
| growing faster | | |
| I notice less shedding/fall out | 91.4% | 100.0% |
| My scalp is less irritated | 80.0% | 86.7% |
| My scalp feel healthier | 97.1% | 100.0% |
| My scalp is less visible | 97.1% | 96.7% |
| I see new hair growth | 97.1% | 100.0% |

### Phototrichogram Analysis—Statistical Significant Results—Hair Treatment Regimen

Pursuant to phototrichogram analysis, a statistically significant improvement in the number of anagen hair, anagen/telogen ratio and total density parameters was observed after 120 and 180 days of regimen use, indicating an improvement in hair growth and density. Thus, a statistically significant improvement in hair growth and hair density was observed after 120 and 180 days of Hair Treatment Regimen use.

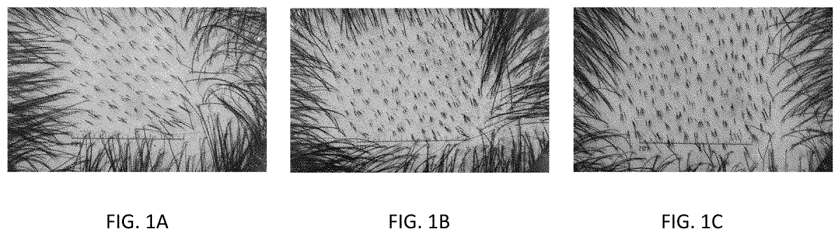

The results of the phototrichogram analysis for the Hair Treatment Regimen are further illustrated in FIGS. 1A-1C, 2A-2C and 3A-3B. FIG. 1A illustrates the baseline anagen hair count (hair count was 138) of an exemplary test subject at initial assessment (T0-D0). FIG. 1B illustrates the anagen hair count (hair count was 216; increase of 78 over baseline) of the same test subject at 120 days (T120). Thus, use of the Hair Treatment Regimen resulted in a 50% of anagen hair after 120 days of daily administration. FIG. 1C illustrates the anagen hair count (hair count was 248; increase of 110 over baseline) of the same test subject at 180 days. Thus, use of the Hair Treatment Regimen resulted in an 80% of anagen hair after 180 days of daily administration. The statistical results for the phototrichogram analysis of anagen hair count for the Hair Treatment Regimen across all test subjects is summarized in Table 7. The statistical results for the phototrichogram analysis of the ratio of anagen phase hair to telogen phase hair for the Hair Treatment Regimen across all test subjects is summarized in Table 8. The statistical results for the phototrichogram analysis of hair density for the Hair Treatment Regimen across all test subjects is summarized in Table 9.

TABLE 7
Anagen Phase Hair Increase for Hair Treatment Regimen

| | T0-D2 | T120-D2 | T180-D2 |
|---|---|---|---|
| Mean | 137.7 | 160.8 | 176.0 |
| Percent (%) increase in | | 16.8% | 27.8% |
| anagen hair | | | |

TABLE 8
Anagen/Telogen Ratio for Hair Treatment Regimen

| | T0-D2 | T120-D2 | T180-D2 |
|---|---|---|---|
| Mean | 1.61 | 1.78 | 2.48 |
| Percent (%) increase in | | 10.6% | 54.0% |
| anagen/telogen ratio | | | |

TABLE 9
Hair Density for Hair Treatment Regimen

| | T0-D2 | T120-D2 | T180-D2 |
|---|---|---|---|
| Mean | 230.3 | 257.3 | 250.3 |
| Percent (%) increase in hair | | 11.7 | 8.7 |
| density | | | |

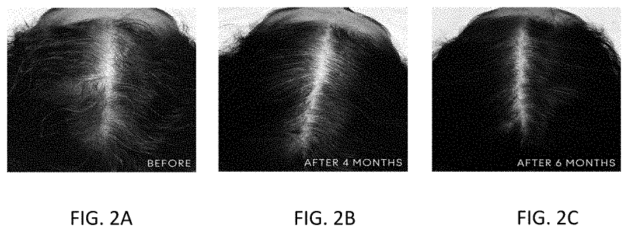

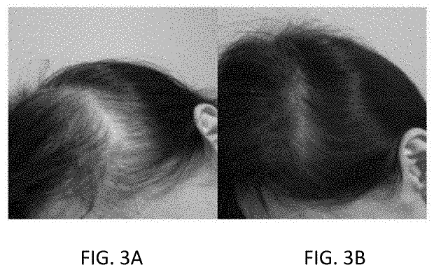

FIGS. 2A and 3A each provide whole head photographic evidence of hair loss of an exemplary test subject at initial assessment (T0-D0). FIG. 2B provides whole head photographic evidence supporting improved hair growth and density of the same exemplary test subject at 120 days (T120) after daily administration of the Hair Treatment Regimen. FIGS. 2C and 3B provide whole head photographic evidence of further improved hair growth and improved hair density of the same exemplary test subject at 180 days (T180) after daily administration of the Hair Treatment Regimen.

### Phototrichogram Analysis—Statistical Significant Results—Minoxidil Regimen

Pursuant to phototrichogram analysis, a statistically significant improvement in the number of anagen hair, anagen/telogen ratio and total density parameters was observed after 120 and 180 days of regimen use, indicating an improvement in hair growth and density. Thus, a statistically significant improvement in hair growth and a statistically significant improvement in hair density was observed after 120 and 180 days of Minoxidil Regimen use.

Thus, the Hair Treatment Regimen was shown to be equally effective as the Minoxidil Regimen at improving the number of anagen hair, anagen/telogen ratio and total density parameters was observed after 120 and 180 days of regimen use.

Self-Assessment Questionnaire—Statistical Significant Results—Hair Treatment Regimen versus Minoxidil Regimen

No statistically significant difference was observed for the Hair Treatment Regimen in comparison with the Minoxidil Regimen at the 90 day (T90) point in the clinical evaluation.

A statistically significant greater agreement was observed for Minoxidil Regimen in comparison with Hair Treatment Regimen at the 120-day (T120) point for statements: “My hair has more volume”, “My hair has more body”, “My scalp feels healthier” and “This regimen is more effective than hair growth solutions I've tried in the past”.

At the 180-day (T180) point, however, there was no statistically significant difference observed for all questionnaire statements assessed. Thus, at T180 day point, the Hair Treatment Regimen was shown to be equally effective as the Minoxidil Regimen based on all questionnaire statements assessed.

### Example 2

### Randomized Clinical Evaluation of Scalp Regimens

The objective of this study was to assess the impact of regimens as provided in Table 10 on scalp pH and microbiome. The shampoo, conditioner and spray of Regimen 1 and Regimen 2 are the formulations set forth in Example 1 (Tables 1 (shampoo), 2 (conditioner) and 3 (spray), respectively). Regimen 3 includes a shampoo, conditioner and spray as set forth in Example 1, however, no keratin protein composition is included in the shampoo, conditioner and spray. Thus, Regimen 3 did not include alpha keratin and gamma keratin proteins.

TABLE 10

| Regimen 1 | Regimen 2 | Regimen 3 |
|---|---|---|
| Shampoo | Shampoo | Shampoo |
| Conditioner | Conditioner | Conditioner |
| | Spray | Spray |

Twelve test subjects utilized Regimen 1 and Regimen 2. Ten subjects utilized Regimen 3. For Regimen 1, each test subject used the Shampoo and Conditioner every other day for 28 days. For Regimen 2, the Shampoo and Conditioner were applied every other day for 28 days while the Spray was applied every day. For Regimen 3, the Shampoo and Conditioner were applied every other day for 28 days while the Spray was applied every day. Application of each formulation was carried out in the same manner as set forth in Example 1.

### pH Analysis

A statistically significant increase in pH over baseline (first day of study) was observed after 28 days of Regimen 1 and Regimen 2. Specifically, as set forth in Table 11, Regimen 1 and Regimen 2 each provided a 10% increase in pH. Such an increase in pH is attributable to the presence of the keratin protein composition that is found in Regimen 1 and Regimen 2. No statistically significant increase in pH over baseline was observed after 28 days of Regimen 3. By increasing pH to a level that remains below 5.5 but above 4.5 as well as above the pH value of hair (about 3.6), the slightly acidic environment created by Regimen 1 and Regimen 2 provided a scalp environment that prevents fungal and bacterial overgrowth while keeping hair follicles relaxed/open and healthy. By maintaining a healthy slightly acidic pH, Regimen 1 and Regimen 2 were also shown to nourish and maintain the natural acidic sebum of the scalp.

TABLE 11
Increase in pH (%) Relative to Baseline

| | |
|---|---|
| Regimen 1 | 10.2% |
| Regimen 2 | 10.2% |
| Regimen 3 | 4.6% |

### Microbiome Analysis

The microbiome of the scalp skin was evaluated using analysis of skin surface samples obtained using Swab Kits (DNA Genotek) that contain sterile cotton swabs, a tube, containing 1.0 mL of NaCl (0.15 M)—Tween 20 (0.1%) solution and a second tube for storage following completion of the swab. Prior to completing the swabbing, each swab was placed in a tube containing 5.0 mL of NaCl (0.15 M)—Tween 20 (0.1%) solution. An IRSI technician performed swabbing of the subjects' scalp skin on the crown of the heat. The swab was put in tubes for shipping according to the procedure in the instructions that come with each swab kit. Comparison analyzes of the microbiome of the study subjects in the three regimens were carried out by Gentros before investigational and control products use (T0) and 28 days after investigational and control products use.

The microbiota analysis was performed both quantitatively (abundance) and qualitatively (diversity). The microbiota readings generated from each subject were compared in existing public databases to classify the species present and the number of times each appears in the subject's microbiome in the collected area. Bioinformatics data was based on statistical analysis to validate and eliminate noise from the analysis. The software used was Qiime2 (https://qiime2.org/).

The readings generated for each species of bacteria found in each study subject were transformed using the logarithmic function to reduce data variation. After this treatment, the data were analyzed by two non-parametric Wilcoxon Signed Rank statistical methods when comparing the samples collected at baseline with samples after 28 days of treatment for the same individual and Mann-Whitney when comparing between the three regimens, since each regimen had a different group of subjects. Only the readings that obtained the 90% and 95% confidence level (10% and 5% significance, p<0.1 and p<0.05 respectively) were considered. The software used was XLSTAT 2021 and MINITAB 14.

An increase in the Cutibacterium (Propionibacterium) genus was observed after 28 days of use of Regimen 1. An increase in the Lawsonella (Corynebacterium) genus was observed after 28 days of use of Regimen 2 and Regimen 3. Once this Lawsonella genus is more common on humid regions than sebaceous, these results indicate an increase in scalp moisturization and a healthier microbiome. Finally, an increase in the number of Pseudomonas genus and a decrease in Streptococcus genus was observed after 28 days of use of Regimen 3. The microbiome diversity was maintained after the use of all regimens.

### GENERALIZED STATEMENTS OF THE DISCLOSURE

The following numbered statements provide a general description of the disclosure and are not intended to limit the appended claims.

Statement 1. A hair treatment composition is provided that includes a keratin protein composition that includes a blend of alpha keratin and gamma keratin.
Statement 2. The present disclosure provides a hair treatment composition of Statement 1, wherein the keratin protein is not denatured and maintains natural keratin protein conformation.
Statement 3. The present disclosure provides a hair treatment composition of Statements 1-2, wherein the keratin protein composition includes at least about 70% w/w alpha keratin protein.
Statement 4. The present disclosure provides a hair treatment composition of Statements 1-3, wherein the keratin protein composition includes at least about 80% w/w alpha keratin protein.
Statement 5. The present disclosure provides a hair treatment composition of Statements 1-4, wherein the keratin protein composition includes at least about 90% w/w alpha keratin protein.
Statement 6. The present disclosure provides a hair treatment composition of Statements 1-5, wherein the keratin protein composition includes at least about 95% w/w alpha keratin protein.
Statement 7. The present disclosure provides a hair treatment composition of Statements 1-6, wherein the keratin protein composition includes at least about 98% w/w alpha keratin protein.
Statement 8. The present disclosure provides a hair treatment composition of Statements 1-7, wherein the keratin protein composition includes at least about 2.0% w/w gamma keratin protein.
Statement 9. The present disclosure provides a hair treatment composition of Statements 1-8, wherein the keratin protein composition includes at least about 5.0% w/w gamma keratin protein.
Statement 10. The present disclosure provides a hair treatment composition of Statements 1-9, wherein the keratin protein composition includes at least about 10.0% w/w gamma keratin protein.
Statement 11. The present disclosure provides a hair treatment composition of Statements 1-10, wherein the keratin protein composition includes at least about 20.0% w/w gamma keratin protein.
Statement 12. The present disclosure provides a hair treatment composition of Statements 1-11, wherein the keratin protein composition includes at least about 30.0% w/w gamma keratin protein.
Statement 13. The present disclosure provides a hair treatment composition of Statements 1-12, wherein at least about 30% of the alpha keratin exhibits a peak average molecular weight (Mp) of at least about 443 kDa.
Statement 14. The present disclosure provides a hair treatment composition of Statements 1-13, wherein the gamma keratin exhibit a peak average molecular weight (Mp) of at least about 15 kDa to about 45 kDa.
Statement 15. The present disclosure provides a hair treatment composition of Statements 1-14, wherein the gamma keratin exhibit a peak average molecular weight (Mp) of at least about 30 kDa.
Statement 16. The present disclosure provides a hair treatment composition of Statements 1-15, wherein the hair treatment composition includes at least one peptide.
Statement 17. The present disclosure provides a hair treatment composition of Statements 1-16, wherein the hair treatment composition includes at least one biomimetic signal peptide.
Statement 18. The present disclosure provides a hair treatment composition of Statements 1-17, wherein the at least one biomimetic signal peptide is acetyl tetrapeptide-3.
Statement 19. The present disclosure provides a hair treatment composition of Statements 1-18, wherein the at least one biomimetic signal peptide is acetyl tetrapeptide-3, biotinoyl tripeptide-1, tripeptide-1, acetyl hexapeptide-8, acetyl dipeptide-1, caproyl tetrapeptide-3, carnosine, glutathione, palmitoyl oligopeptide, palmitoyl tetrapeptide-7, acetyl tetrapeptide-5, palmitoyl hexapeptide-14, pentapeptide-3, nonapeptide-1, acetyl hexapeptide, hexapeptide-11, hexanoyl dipeptide-3, acetyl octapeptide-3, palmitoyl tripeptide-5, palmitoyl dipeptide-5, palmitoyl dipeptide-6, acetyl tetrapetide-2, myristoyl pentapeptide-17, or any combination thereof.
Statement 20. The present disclosure provides a hair treatment composition of Statements 1-19, wherein the hair treatment composition includes at least one clover extract.
Statement 21. The present disclosure provides a hair treatment composition of Statements 1-20, wherein the at least one clover extract is a red clover flower extract (Trifolium pratense L.).
Statement 22. The present disclosure provides a hair treatment composition of Statements 1-21, wherein the hair treatment composition includes at least one probiotic.
Statement 23. The present disclosure provides a hair treatment composition of Statements 1-22, wherein the hair treatment composition includes at least one active green tea or green tea extract.
Statement 24. The present disclosure provides a hair treatment composition of Statements 1-23, wherein the hair treatment composition includes at least one rice-based humectant.
Statement 25. The present disclosure provides a hair treatment composition of Statements 1-24, wherein the hair treatment composition includes a complex comprising: a keratin protein composition comprising alpha keratin, gamma keratin; at least one biomimetic signal peptide; and red clover extract.
Statement 26. The present disclosure provides a hair treatment composition of Statements 1-25, wherein the hair treatment composition is color safe.
Statement 27. The present disclosure provides a hair treatment composition of Statements 1-26, wherein the hair treatment composition is paraben-free, sulfate-free, vegan and gluten-free.
Statement 28. The present disclosure provides a hair treatment composition of Statements 1-27, wherein the hair treatment composition is free of active pharmaceutical ingredients.
Statement 29. The present disclosure provides a hair treatment composition of Statements 1-28, wherein the hair treatment composition includes at least one active pharmaceutical ingredient for supporting hair growth.
Statement 30. The present disclosure provides a hair treatment composition of Statements 1-29, wherein the at least one active pharmaceutical ingredient for supporting hair growth is minoxidil.
Statement 31. The present disclosure provides a hair treatment composition of Statements 1-30, wherein the minoxidil supports new hair growth.
Statement 32. The present disclosure provides a hair treatment composition of Statements 1-31, wherein the minoxidil supports existing hair growth.
Statement 33. The present disclosure provides a hair treatment composition of Statements 1-32, wherein the minoxidil is present at a concentration of about 0.1% w/w to about 15% w/w.
Statement 34. The present disclosure provides a hair treatment composition of Statements 1-33, wherein the minoxidil is present at a concentration of about 5% w/w.
Statement 35. The present disclosure provides a hair treatment composition of Statements 1-34, formulated as a shampoo, conditioner, aqueous solution, powder, gel, mousse/foam, spray, dressing, lotion, wax, hydrogel, oil, emulsion, paste, polish or cream.
Statement 36. The present disclosure provides a hair treatment composition of Statements 1-35, wherein the shampoo includes a keratin protein composition as provided herein, water, red clover extract, sodium hyaluronate, red algae extract, rice oil glycereth-8 esters, and at least one biomimetic signal peptide such as acetyl tetrapeptide-3.
Statement 37. The present disclosure provides a hair treatment composition of Statements 1-36, wherein the conditioner includes a keratin protein composition, water, red clover extract, at least one biomimetic signal peptide such as acetyl tetrapeptide-3, Cyperus esculentus root oil, and at least one probiotic such as saccharomyces lysate extract.
Statement 38. The present disclosure provides a hair treatment composition of Statements 1-37, wherein the spray includes a keratin protein composition, water, red clover extract, at least one biomimetic signal peptide, at least one probiotic such as saccharomyces lysate extract, larix/larch europaea wood extract, and methyl nicotinate.
Statement 39. The present disclosure provides a hair treatment composition of Statements 1-38, further includes one or more of water, cetearyl alcohol, dimethicone, brassicamidopropyl dimethylamine, cetyl esters, cetyl alcohol, behentrimonium chloride, isododecane, hydroxyethylcellulose, polysorbate 60, benzyl alcohol, sorbic acid, benzoic acid, glycerin, phenoxyethanol, dimethiconol, chlorphenesin, gluconolactone, ethylhexylglycerin, PGE-100 stearate, glyceryl stearate, Silybum marianum, ethyl ester, tocopheryl nicotinate, Cyperus esculentus root oil, madecassoside, Quercus Patrea fruit extract, hydrolyzed quinoa, Oryza sativa (rice) extract, Rosmarinus officinalis (Rosemary) leaf extract, hydrogenated ethylhexyl olivate, Oryza sativa (rice) bran extract, hydrogenated olive oil unsaponifiables, jojoba esters, Helianthus annuus (sunflower) seed oil, Oryza sativa (rice) seed protein, phytic acid, tetrasodium glutamate diacetate, methyl nicotinate, acetyl tetrapeptide-3, sodium lauroyl methyl isethionate, sodium cocoyl isethionate, lauryl glucoside, acrylates copolymer, sodium hyaluronate, rice oil, glycereth-8 esters, cocamide MIPA, polysorbate 20, hydrogenated coconut acid, sodium isethionate, sodium methyl cocoyl taurate, caprylyl glycol, guar hydroxypropyltrimonium chloride, Chondrus crispus extract, ethylhexyl salicylate, sodium hydroxide, sodium chloride, Zingiber officinale (ginger) root extract, Pisum sativum (Pea) extract, Nasturtium Officinale extract, Moringa Oleifera seed extract, Jasminum officinale (Jasmine) flower extract, hydrolyzed linseed extract, hydrolyzed Hibiscus Esculentus extract, Helianthus annuus (sunflower) seed extract, Daucus carota sativa (Carrot) root extract, Cynara scolymus (artichoke) leaf extract, Amaranthus caudatus seed extract, Saccharomyces Lysate extract (probiotic yeast for increasing oxygenation and reducing irritation in the scalp), Larix europaea wood extract, Trifolium pratense (clover) flower extract, Camellia sinensis leaf extract, Isochrysis galbana extract, tocopherol, Mentha piperita (Peppermint) oil, pentylene glycol, sodium phytate, sodium dilauramidoglutamide lysine, quaternium-51, ethylhexylglycerin, sodium metabisulfite, glycine, a fragrance, biosaccharide gum-4, butylene glycol, glycerin, zinc chloride, PPG-26-Buteth-26, propanediol, polyacrylate crosspolymer-6, PEG-40 hydrogenated castor oil, t-butyl alcohol, alcohol, sodium cocoyl glutamate, tetrasodium EDTA, trisodium ethylenediamine disuccinate, butylene glycol, polyglyceryl-6 oleate, glyceryl caprylate, sodium dilauramidoglutamide lysine, caprylyl/capryl glucoside, ethylhexylglycerin, sodium benzoate, potassium sorbate, disodium EDTA, phenoxyethanol, sodium surfactin, dextran, citric acid, linalool, geraniol, limonene, citronellol, benzyl benzoate, citral, hexyl cinnamal, benzophenone-4, benzophenoneo-3, or any combination thereof.
Statement 40. The present disclosure provides a hair treatment composition of Statements 1-39, wherein the hair treatment composition is sulfate-free.
Statement 41. The present disclosure provides a hair treatment composition of Statements 1-40, wherein the hair treatment composition cleanses a hair shaft and clears debris from follicle openings, creating an environment for healthy hair growth.
Statement 42. A method of supporting new hair growth is provided that includes the step of: administering an effective amount of a hair treatment composition as provided herein to a surface of the hair and scalp in need of treatment.
Statement 43. A method of supporting existing hair growth is provided that includes the step of: administering an effective amount of a hair treatment composition as provided herein to a surface of the hair and scalp in need of treatment.
Statement 44. A method for the treatment or management of alopecia is provided that includes the step of administering to a subject having alopecia an effective amount of a hair treatment composition as provided herein.
Statement 45. The present disclosure provides a method of Statement 44, wherein the alopecia is attributed to or caused by hereditary factors, hormonal fluctuations, recent pregnancy, nutritional deficiencies, stress, or any combination thereof.
Statement 46. The present disclosure provides a method of Statements 44-45, wherein the alopecia is androgenetic alopecia.
Statement 47. The present disclosure provides a method of Statements 44-46, wherein the alopecia is androgenetic alopecia of a female human.
Statement 48. The present disclosure provides a method of Statements 44-47, wherein the alopecia is androgenetic alopecia of a female human's scalp.
Statement 49. A method for the treatment or management of telogen effluvium is provided that includes the step of: administering an effective amount of a hair treatment composition as provided herein to a surface of the hair and scalp in need of treatment.
Statement 50. A method of increasing hair thickness is provided includes the step of: administering an effective amount of a hair treatment composition as provided herein to a surface of the hair and scalp in need of treatment.
Statement 51. The present disclosure provides a method of Statement 50, wherein the hair treatment composition clears hair follicles and strengthens a moisture barrier on the scalp to support improved hair growth.
Statement 52. A method of decreasing hair thinning is provided that includes the step of: administering an effective amount of a hair treatment composition as provided herein to a surface of the hair and scalp in need of treatment.
Statement 53. The present disclosure provides a method of Statement 52, wherein the thinning hair is due to aging.
Statement 54. A method of decreasing hair shedding is provided that includes the step of: administering an effective amount of a hair treatment composition as provided herein to a surface of the hair and scalp in need of treatment.
Statement 55. The present disclosure provides a method of Statement 54, wherein the hair treatment composition is a shampoo as provided herein.
Statement 56. The present disclosure provides a method of Statements 54-55, wherein the method further includes the steps of administering an effective amount of a conditioner as provided herein and administering an effective amount of a spray or foam as provided herein.
Statement 57. A method of decreasing hair loss is provided that includes the step of: administering an effective amount of a hair treatment composition as provided herein to a surface of the hair and scalp in need of treatment.
Statement 58. The present disclosure provides a method of Statement 57, wherein the hair treatment composition is a shampoo as provided herein.
Statement 59. The present disclosure provides a method of Statement 57-58, wherein the method further includes the steps of administering an effective amount of a conditioner as provided herein and administering an effective amount of a spray or foam as provided herein.
Statement 60. A method of increasing anagen hair count is provided that includes the step of administering an effective amount of a hair treatment composition as provided herein to a surface of the hair and scalp in need of treatment, wherein the hair treatment composition is formulated as a shampoo.
Statement 61. The present disclosure provides a method of Statement 60, wherein the anagen hair count is increased by at least 5%, 10%, 15%, 20%, 25%, 30%, 35%, 40%, 45%, or 50% after 120 days of daily administration.
Statement 62. The present disclosure provides a method of Statements 60-61, wherein the anagen hair count is increased by at least 50% after 120 days of daily administration.
Statement 63. The present disclosure provides a method of Statements 60-62, wherein the anagen hair count is increased by at least 5%, 10%, 15%, 20%, 25%, 30%, 35%, 40%, 45%, or 50%, 55%, 60%, 65%, 70%, 75%, or 80% after 180 days of daily administration
Statement 64. The present disclosure provides a method of Statement 60-63, wherein the anagen hair count is increased by at least 80% after 180 days of daily administration.
Statement 65. A method of increasing anagen hair count is provided that includes the step of administering an effective amount of a hair treatment composition as provided herein to a surface of the hair and scalp in need of treatment, wherein the hair treatment composition is formulated as a conditioner.
Statement 66. The present disclosure provides a method of Statement 65, wherein the anagen hair count is increased by at least 5%, 10%, 15%, 20%, 25%, 30%, 35%, 40%, 45%, or 50% after 120 days of daily administration.
Statement 67. The present disclosure provides a method of Statements 65-66, wherein the anagen hair count is increased by at least 50% after 120 days of daily administration.
Statement 68. The present disclosure provides a method of Statements 65-67, wherein the anagen hair count is increased by at least 5%, 10%, 15%, 20%, 25%, 30%, 35%, 40%, 45%, or 50%, 55%, 60%, 65%, 70%, 75%, or 80% after 180 days of daily administration
Statement 69. The present disclosure provides a method of Statements 65-68, wherein the anagen hair count is increased by at least 80% after 180 days of daily administration.
Statement 70. A method of increasing anagen hair count is provided that includes the step of administering an effective amount of a hair treatment composition as provided herein to a surface of the hair and scalp in need of treatment, wherein the hair treatment composition is formulated as a spray.
Statement 71. The present disclosure provides a method of Statement 70, wherein the anagen hair count is increased by at least 5%, 10%, 15%, 20%, 25%, 30%, 35%, 40%, 45%, or 50% after 120 days of daily administration.
Statement 72. The present disclosure provides a method of Statements 70-71, wherein the anagen hair count is increased by at least 50% after 120 days of daily administration.
Statement 73. The present disclosure provides a method of Statements 70-72, wherein the anagen hair count is increased by at least 5%, 10%, 15%, 20%, 25%, 30%, 35%, 40%, 45%, or 50%, 55%, 60%, 65%, 70%, 75%, or 80% after 180 days of daily administration
Statement 74. The present disclosure provides a method of Statements 70-73, wherein the anagen hair count is increased by at least 80% after 180 days of daily administration.
Statement 75. A method of increasing anagen hair count is provided that includes the step of administering an effective amount of a hair treatment composition as provided herein to a surface of the hair and scalp in need of treatment, wherein the hair treatment composition is formulated as a foam.
Statement 76. The present disclosure provides a method of Statement 75, wherein the anagen hair count is increased by at least 5%, 10%, 15%, 20%, 25%, 30%, 35%, 40%, 45%, or 50% after 120 days of daily administration.
Statement 77. The present disclosure provides a method of Statements 75-76, wherein the anagen hair count is increased by at least 50% after 120 days of daily administration.
Statement 78. The present disclosure provides a method of Statements 75-77, wherein the anagen hair count is increased by at least 5%, 10%, 15%, 20%, 25%, 30%, 35%, 40%, 45%, or 50%, 55%, 60%, 65%, 70%, 75%, or 80% after 180 days of daily administration
Statement 79. The present disclosure provides a method of Statements 75-78, wherein the anagen hair count is increased by at least 80% after 180 days of daily administration.
Statement 80. A method of increasing anagen hair count is provided that includes the steps of: administering an effective amount of a first hair treatment composition as provided herein to a surface of the hair and scalp in need of treatment; administering an effective amount of a second hair treatment composition as provided herein to a surface of the hair and scalp in need of treatment; and administering an effective amount of a third hair treatment composition as provided herein to a surface of the hair and scalp in need of treatment, wherein the first hair treatment condition is formulated as a shampoo, wherein the second hair treatment composition is formulated as a conditioner, and wherein the third hair treatment composition is formulated as a spray.
Statement 81. The present disclosure provides a method of Statement 80, wherein the anagen hair count is increased by at least 5%, 10%, 15%, 20%, 25%, 30%, 35%, 40%, 45%, or 50% after 120 days of daily administration of the first, second and third hair treatment composition.
Statement 82. The present disclosure provides a method of Statements 80-81, wherein the anagen hair count is increased by at least 50% after 120 days of daily administration of the first, second and third hair treatment composition.
Statement 83. The present disclosure provides a method of Statements 80-82, wherein the anagen hair count is increased by at least 5%, 10%, 15%, 20%, 25%, 30%, 35%, 40%, 45%, or 50%, 55%, 60%, 65%, 70%, 75%, or 80% after 180 days of daily administration of the first, second and third hair treatment composition.
Statement 84. The present disclosure provides a method of Statements 80-83, wherein the anagen hair count is increased by at least 80% after 180 days of daily administration of the first, second and third hair treatment composition.
Statement 85. A method of increasing or otherwise promoting hair density is provided that includes the step of: administering an effective amount of a hair treatment composition as provided herein to a surface of the hair and scalp in need of treatment, wherein the hair treatment composition is formulated as a shampoo, conditioner, spray or foam.
Statement 86. The present disclosure provides a method of Statement 85, wherein the hair density is increased by at least about 5%, 10%, 15%, 20%, 25%, 30%, 35%, 40%, 45%, or 50% after 120 days of daily administration.
Statement 87. The present disclosure provides a method of Statements 85-86, wherein the hair density is increased by at least about 5%, 10%, 15%, 20%, 25%, 30%, 35%, 40%, 45%, or 50% after 180 days of daily administration.
Statement 88. The present disclosure provides a method of Statements 85-87, wherein the hair density is increased by at least about 10% after 120 days of daily administration.
Statement 89. The present disclosure provides a method of Statements 85-88, wherein the hair density is increased by at least about 10% after 180 days of daily administration.
Statement 90. A method of increasing hair density is provided that includes the steps of: administering an effective amount of a first hair treatment composition as provided herein to a surface of the hair and scalp in need of treatment; administering an effective amount of a second hair treatment composition as provided herein to a surface of the hair and scalp in need of treatment; and administering an effective amount of a third hair treatment composition as provided herein to a surface of the hair and scalp in need of treatment, wherein the first hair treatment condition is formulated as a shampoo, wherein the second hair treatment composition is formulated as a conditioner, and wherein the third hair treatment composition is formulated as a spray.
Statement 91. The present disclosure provides a method of Statement 90, wherein the hair density is increased by at least about 5%, 10%, 15%, 20%, 25%, 30%, 35%, 40%, 45%, or 50% after 120 days of daily administration.
Statement 92. The present disclosure provides a method of Statements 90-91, wherein the hair density is increased by at least about 5%, 10%, 15%, 20%, 25%, 30%, 35%, 40%, 45%, or 50% after 180 days of daily administration.
Statement 93. The present disclosure provides a method of Statements 90-92, wherein the hair density is increased by at least about 10% after 120 days of daily administration.
Statement 94. The present disclosure provides a method of Statements 90-93, wherein the hair density is increased by at least about 10% after 180 days of daily administration.
Statement 95. A method of increasing the ratio of anagen phase hair to telogen phase hair in a subject in need of treatment is provided that includes the step of administering an effective amount of one or more hair treatment composition as provided in Statements Y to a surface of the hair and scalp in need of treatment.
Statement 96. The present disclosure provides a method of Statement 95, wherein the hair treatment composition increases the ratio of anagen phase hair to telogen phase hair by at least about 5%, 10%, 15%, 20%, 25%, 30%, 35%, 40%, 45%, or 50% after 120 days of daily administration.
Statement 97. The present disclosure provides a method of Statements 95-96, wherein the hair treatment composition increases the ratio of anagen phase hair to telogen phase hair by at least about 5%, 10%, 15%, 20%, 25%, 30%, 35%, 40%, 45%, 50% or 55% after 180 days of daily administration.
Statement 98. A hair treatment regimen is provided that includes the steps of: administering an effective amount of a first hair treatment composition as provided herein to a surface of the hair and scalp in need of treatment; administering an effective amount of a second hair treatment composition as provided herein to a surface of the hair and scalp in need of treatment; and administering an effective amount of a third hair treatment composition as provided herein to a surface of the hair and scalp in need of treatment, wherein the first hair treatment condition is formulated as a shampoo, wherein the second hair treatment composition is formulated as a conditioner, and wherein the third hair treatment composition is formulated as a spray.
Statement 99. The present disclosure provides a method of Statement 98, wherein the shampoo includes a keratin protein composition as provided herein, water, red clover extract, sodium hyaluronate, red algae extract, rice oil glycereth-8 esters, and at least one biomimetic signal peptide such as acetyl tetrapeptide-3.
Statement 100. The present disclosure provides a method of Statements 98-99, wherein the conditioner includes a keratin protein composition, water, red clover extract, at least one biomimetic signal peptide such as acetyl tetrapeptide-3, Cyperus esculentus root oil, and at least one probiotic such as saccharomyces lysate extract.
Statement 101. The present disclosure provides a method of Statements 98-100, wherein the spray includes a keratin protein composition, water, red clover extract, at least one biomimetic signal peptide, at least one probiotic such as saccharomyces lysate extract, larix/larch europaea wood extract, and methyl nicotinate.
Statement 102. The present disclosure provides a regimen of Statements 98-101, wherein the anagen hair count is increased by at least 5%, 10%, 15%, 20%, 25%, 30%, 35%, 40%, 45%, or 50% after 120 days of daily administration of the first, second and third hair treatment composition.
Statement 103. The present disclosure provides a regimen of Statements 98-102, wherein the anagen hair count is increased by at least 5%, 10%, 15%, 20%, 25%, 30%, 35%, 40%, 45%, or 50%, 55%, 60%, 65%, 70%, 75%, or 80% after 180 days of daily administration of the first, second and third hair treatment composition.
Statement 104. A method of supporting, improving or maintaining scalp microbiome is provided that includes the steps of administering an effective amount of a hair treatment composition as provided herein to a surface of a scalp in need of treatment.
Statement 105. The present disclosure provides a method of Statement 104, wherein the hair treatment composition reduces any existing scalp irritation.
Statement 106. The present disclosure provides a method of Statements 104-105, wherein the hair treatment composition is a shampoo as provided herein.
Statement 107. The present disclosure provides a method of Statements 104-106, wherein the method further includes the steps of administering an effective amount of a conditioner as provided herein and administering an effective amount of a spray or foam as provided herein.
Statement 108. A method of reducing scalp irritation is provided that includes the steps of administering an effective amount of a hair treatment composition as provided herein to a surface of a scalp in need of treatment, wherein upon administration scalp irritation is reduced.
Statement 109. The present disclosure provides a method of Statement 108, wherein the hair treatment composition is a shampoo as provided herein.
Statement 110. The present disclosure provides a method of Statements 108-109, wherein the method further includes the steps of administering an effective amount of a conditioner as provided herein and administering an effective amount of a spray or foam as provided herein.
Statement 111. A method for reducing hair breakage is provided that includes the step of: administering an effective amount of a hair treatment composition as provided herein to a surface of the hair and scalp in need of treatment.
Statement 112. The present disclosure provides a method of Statement 111, wherein the hair breakage is reduced by at least 20%, 30%, 40%, 50% or more after one administration.
Statement 113. A method of reducing hair frizziness is provided that includes the step of: administering an effective amount of a hair treatment composition as provided herein to a surface of the hair and scalp in need of treatment.
Statement 114. The present disclosure provides a method of Statement 113, wherein the hair frizziness is reduced by at least 20%, 30%, 40%, 50% or more after one administration.
Statement 115. A method of improving scalp flora ecology in a subject is provided that includes the step of administering, applying or otherwise introducing an effective amount of at least one hair treatment composition to a surface of the scalp in need of treatment.
Statement 116. The present disclosure provides a method of Statement 115, wherein the at least one hair treatment composition is a shampoo, conditioner, spray, foam or any combination thereof.
Statement 117. The present disclosure provides a method of Statements 115-116, wherein the shampoo includes a keratin protein composition as provided herein, water, red clover extract, sodium hyaluronate, red algae extract, rice oil glycereth-8 esters, and at least one biomimetic signal peptide such as acetyl tetrapeptide-3.
Statement 118. The present disclosure provides a method of Statements 115-117, wherein the conditioner includes a keratin protein composition, water, red clover extract, at least one biomimetic signal peptide such as acetyl tetrapeptide-3, Cyperus esculentus root oil, and at least one probiotic such as saccharomyces lysate extract.
Statement 119. The present disclosure provides a method of Statements 115-118, wherein the spray includes a keratin protein composition, water, red clover extract, at least one biomimetic signal peptide, at least one probiotic such as saccharomyces lysate extract, larix/larch europaea wood extract, and methyl nicotinate.
Statement 120. A method of maintaining scalp microbiome diversity in a subject is provided that includes the step of administering, applying or otherwise introducing an effective amount of at least one hair treatment composition to a surface of the scalp in need of treatment.
Statement 121. The present disclosure provides a method of Statement 120, wherein the at least one hair treatment composition is a shampoo, conditioner, spray, foam or any combination thereof.
Statement 122. The present disclosure provides a method of Statements 120-121, wherein the shampoo includes a keratin protein composition as provided herein, water, red clover extract, sodium hyaluronate, red algae extract, rice oil glycereth-8 esters, and at least one biomimetic signal peptide such as acetyl tetrapeptide-3.
Statement 123. The present disclosure provides a method of Statements 120-122, wherein the conditioner includes a keratin protein composition, water, red clover extract, at least one biomimetic signal peptide such as acetyl tetrapeptide-3, Cyperus esculentus root oil, and at least one probiotic such as saccharomyces lysate extract.
Statement 124. The present disclosure provides a method of Statements 120-123, wherein the spray includes a keratin protein composition, water, red clover extract, at least one biomimetic signal peptide, at least one probiotic such as saccharomyces lysate extract, larix/larch europaea wood extract, and methyl nicotinate.
Statement 125. A method of increasing in the Lawsonella (Corynebacterium) genus is provided that includes the step of administering, applying or otherwise introducing an effective amount of at least one hair treatment composition to a surface of the scalp in need of treatment.
Statement 126. The present disclosure provides a method of Statement 125, wherein the at least one hair treatment composition is a shampoo, conditioner, spray, foam or any combination thereof.
Statement 127. The present disclosure provides a method of Statements 125-126, wherein the shampoo includes a keratin protein composition as provided herein, water, red clover extract, sodium hyaluronate, red algae extract, rice oil glycereth-8 esters, and at least one biomimetic signal peptide such as acetyl tetrapeptide-3.
Statement 128. The present disclosure provides a method of Statements 125-127, wherein the conditioner includes a keratin protein composition, water, red clover extract, at least one biomimetic signal peptide such as acetyl tetrapeptide-3, Cyperus esculentus root oil, and at least one probiotic such as saccharomyces lysate extract.
Statement 129. The present disclosure provides a method of Statements 125-128, wherein the spray includes a keratin protein composition, water, red clover extract, at least one biomimetic signal peptide, at least one probiotic such as saccharomyces lysate extract, larix/larch europaea wood extract, and methyl nicotinate.

All publications, patents and patent applications cited in this specification are incorporated herein by reference for the teaching to which such citation is used.

The specific responses observed may vary according to and depending on the particular active compound selected or whether there are present carriers, as well as the type of formulation and mode of administration employed, and such expected variations or differences in the results are contemplated in accordance with practice of the present invention.

Although specific embodiments of the present invention are herein illustrated and described in detail, the invention is not limited thereto. The above detailed descriptions are provided as exemplary of the present invention and should not be construed as constituting any limitation of the invention. Modifications will be obvious to those skilled in the art, and all modifications that do not depart from the spirit of the invention are intended to be included with the scope of the appended claims.

1. A hair treatment composition comprising:
a keratin protein composition comprising alpha keratin and gamma keratin;
at least one biomimetic signal peptide; and
red clover extract.

2. The hair treatment composition of claim 1, wherein the keratin protein composition comprises about 95% w/w alpha keratin and about 5% w/w gamma keratin.

3. The hair treatment composition of claim 1, wherein at least about 30% of the alpha keratin exhibits a peak average molecular weight (Mp) of at least about 443 kDa.

4. The hair treatment composition of claim 1, wherein the gamma keratin exhibits a peak average molecular weight (Mp) of from at least about 15 kDa to about 45 kDa.

5. The hair treatment composition of claim 1, wherein the at least one biomimetic signal peptide is selected from the group consisting of acetyl tetrapeptide-3, biotinoyl tripeptide-1, tripeptide-1, acetyl hexapeptide-8, acetyl dipeptide-1, caproyl tetrapeptide-3, carnosine, glutathione, palmitoyl oligopeptide, palmitoyl tetrapeptide-7, acetyl tetrapeptide-5, palmitoyl hexapeptide-14, pentapeptide-3, nonapeptide-1, acetyl hexapeptide, hexapeptide-11, hexanoyl dipeptide-3, acetyl octapeptide-3, palmitoyl tripeptide-5, palmitoyl dipeptide-5, palmitoyl dipeptide-6, acetyl tetrapetide-2, and myristoyl pentapeptide-17.

6. The hair treatment composition of claim 1, further comprising one or more of at least one probiotic, at least one active green tea or green tea extract, at least one rice-based humectant, and panthenol.

7. The hair treatment composition of claim 1, formulated as a shampoo, conditioner, spray, or foam.

8. A method of increasing anagen hair count comprising the steps of:
administering an effective amount of a first hair treatment composition formulated as a shampoo to a surface of the hair and scalp in need of treatment;
administering an effective amount of a second hair treatment composition formulated as a conditioner to a surface of the hair and scalp in need of treatment; and
administering an effective amount of a third hair treatment composition formulated as a spray to a surface of the hair and scalp in need of treatment,
wherein each of the first, second, and third hair treatment composition comprise:
a keratin protein composition comprising alpha keratin, gamma keratin;
at least one biomimetic signal peptide; and
red clover extract.

9. The method of claim 8, wherein the shampoo further comprises sodium hyaluronate, red algae extract, and rice oil glycereth-8 esters.

10. The method of claim 8, wherein the conditioner further comprises acetyl tetrapeptide-3, Cyperus esculentus root oil, and at least one probiotic.

11. The method of claim 8, wherein the spray further comprises at least one probiotic, larix/larch europaea wood extract, and methyl nicotinate.

12. The method of claim 8, wherein anagen hair count is increased by at least 15% after 120 days of daily administration of the first, second and third hair treatment composition.

13. The method of claim 8, wherein anagen hair count is increased by at least 25% after 180 days of daily administration of the first, second and third hair treatment composition.

14. A method of increasing hair density comprising the steps of:
administering an effective amount of a first hair treatment composition formulated as a shampoo to a surface of the hair and scalp in need of treatment;
administering an effective amount of a second hair treatment composition formulated as a conditioner to a surface of the hair and scalp in need of treatment; and
administering an effective amount of a third hair treatment composition formulated as a spray to a surface of the hair and scalp in need of treatment,
wherein each of the first, second, and third hair treatment composition comprise:
a keratin protein composition comprising alpha keratin, gamma keratin;
at least one biomimetic signal peptide; and
red clover extract.

15. The method of claim 14, wherein hair density is increased by at least 10% after 120 days of daily administration of the first, second and third hair treatment composition.

16. A method of promoting scalp microbiome health in a subject comprising the step of:
administering an effective amount of a hair treatment composition to a surface of the scalp in need of treatment,
wherein the hair treatment composition increases the growth of beneficial microorganisms on the scalp and inhibits the growth of harmful microorganisms on the scalp, and
wherein the hair treatment composition comprises:
a keratin protein composition comprising alpha keratin, gamma keratin;
at least one biomimetic signal peptide; and
red clover extract.

17. The method of claim 16, wherein the pH of the scalp is increased by at least about 10% after 28 days of daily administration.

18-20. (canceled)
